SEQUENCE LISTING (7 sequences)

SEQ ID NO: 1 - protein, 4 aa, Artificial Sequence
  Synthetic construct
LLVY

SEQ ID NO: 2 - protein, 75 aa, Saccharomyces cerevisiae
TTTLAFRFQGGIIVAVDSRATAGNWVASQTVKKVIEINPFLLGTMAGGAADCQFWETWLGSQCRLHELREKERIS

SEQ ID NO: 3 - protein, 75 aa, Salinispora tropica
TTIVAATFAGGVLLAGDRRTTMGNLIAGRDVDKLTITDDYSAVGFAGTVGISIDMTRLFVVELEHYEKIEGVPLS

SEQ ID NO: 4 - protein, 75 aa, Frankia sp.
TTIVAVAFPGGVIMAGDRRATQGHMIAQRDVEKVHHADEFSCVGYAGTAGVGAELIRLFQVELEHYEKIEGSTLS

SEQ ID NO: 5 - protein, 75 aa, Salinispora tropica
TTIVAIATAGGVVLAGDRRATMGNLIAQRDVEKVHPADAYSLVGMAGAAGIGIELTRLFQVELEHYEKTEGAMLS

SEQ ID NO: 6 - protein, 75 aa, Streptomyces avermitilis
TTIVATTFPGGVVLAGDRRATMGNVIAQRDIEKVFPADEYSAVGIAGTAGLAVEMVKLFQLELEHFEKVEGATLS

SEQ ID NO: 7 - protein, 75 aa, Streptomyces coelicolor
TTIVAVTFPGGVVLAGDRRATMGNMIAQRDIEKVFPADEYSAVGIAGTAGLAVEMVKLFQLELEHFEKVEGAQLS

(19) United States
(12) Patent Application Publication
(10) Pub. No.: US 2009/0325208 A1
(43) Pub. Date: Dec. 31, 2009

(54) Biosynthesis of Salinosporamide A and Analogs and Methods Thereof

(76) Inventors: Bradley S. Moore, La Jolla, CA (US); Laura Beer, Tucson, AZ (US); Alessandra Eustaquio, Solana Beach, CA (US)

Correspondence Address: DLA PIPER LLP (US), 4365 EXECUTIVE DRIVE, SUITE 1100, SAN DIEGO, CA, 92121-2133, US

(21) Appl. No.: 12/306,210

(22) PCT Filed: Jun. 26, 2007
(86) PCT No.: PCT/US07/14895

§ 371 Date: Jun. 3, 2009

Related U.S. Application Data
(60) Provisional application No. 60/816,753, filed on Jun. 26, 2006.

(51) Int. Cl. C12Q 1/37; C12P 17/16

(52) U.S. Cl. 435/23; 435/118

(57) ABSTRACT

The present invention relates to a sahnosporamide A composition and methods of making salinosporamide A and analogs thereof. The present invention also relates to methods of identifying 2OS proteasome inhibiting agents.

### FIELD OF THE INVENTION

The present invention relates to Salinosporamide A and analog containing composition and a method of making Salinosporamide A and analogs thereof.

### BACKGROUND INFORMATION

The potent 20S proteasome inhibitor salinosporamide A is produced by the marine bacterium Salinispora tropica, strains CNB476 and CNB440. Salinosporamide A is produced in fermentation by the bacterium Salinispora tropica.

### SUMMARY OF THE INVENTION

The present invention relates to chemical compounds, in particular, heterocyclic compounds such as salinosporamide A and analogs thereof, and to methods of making salinosporamide A and analogs thereof.

In one aspect, the invention provides compositions containing a substantially purified salinosporamide composition, containing a first short chain fatty acid, a second short chain fatty acid, and a proteinogenic or non-proteinogenic amino acid, wherein the relative yield of the composition is substantially free of contaminants. In one embodiment, the invention provides compositions containing a substantially purified salinosporamide a composition, containing acetate, a 5′-chloro-5′-deoxyadenosine (ClDA)-derived intermediate, and a non-proteinogenic amino acid, wherein the relative yield of the composition is substantially free of contaminants.

In another aspect, the invention provides a method of identifying a 20S proteasome resistant salinosporamide or an analog thereof, by contacting a 20S proteasome and a salinosporamide or an analog thereof in the presence of a cleavable substrate, wherein the substrate comprises a cleavable product, wherein the 20S proteasome has proteasome activity, wherein the proteasome cleaves the cleavable product from the substrate in the presence or absence of a 20S proteasome inhibitor; and detecting change in the proteasome activity by measuring the cleavable product, thereby identifying a proteasome resistant salinosporamide and/or analog thereof.

In another embodiment, the invention provides a method of producing a salinosporamide from a transformed bacterium, by introducing a transgene which disrupts or interferes with expression of salinosporamide, and cross-breeding transgene-positive progeny with each other to obtain further transgene-positive progeny.

The present invention also relates to a method of manufacturing a substantially pure salinosporamide derivative by combining a first short chain fatty acid, a second short chain fatty acid, and a proteinogenic or non-proteinogenic amino acid, wherein the relative yield of the composition is substantially free of contaminants.

The present invention also relates to a method of manufacturing a substantially pure salinosporamide a derivative comprising, combining acetate, a ClDA-derived intermediate and a non-proteinogenic amino acid, wherein the relative yield of the composition is substantially free of contaminants.

The present invention also relates to a method of producing salinosporamide from a transformed bacterium, by introducing a transgene which disrupts or interferes with expression of salinosporamide, and cross-breeding transgene-positive progeny with each other to obtain further transgene-positive progeny.

The present invention also relates to a method of producing high-titer recombinant salinosporamide A or an analog thereof, by simultaneously co-infecting a cell with a nucleic acid encoding salinosporamide A or an analog thereof operably linked to a promoter; incubating and growing the cell under suitable conditions; and collecting the salinosporamide A or an analog thereof from the co-infected cell, thereby producing a high-titer.

### BRIEF DESCRIPTION OF THE DRAWINGS

FIG. 1 shows structures of the salinosporamides, omuralide and lactacystin.

FIG. 2 is a schematic representation showing a mechanism of action of γ-lactam-β-lactone agents omuralide and salinosporamide against the 20S proteasome and reactivity of the N-terminal nucleophilic threonine residue.

FIG. 3 shows structures of anti-configured β-lactone-containing natural lipids.

FIG. 4 is a schematic representation showing the observed 13C-labeling patterns in the salinosporamides. The bold lines in salinosporamides A and B represent biosynthetic building blocks derived from [U-13C]glucose. In the case of salinosporamide A, two labeling patterns emerged in the cyclohexene ring and the chloroethyl substituted γ-lactam consisting of an intact 4-carbon unit (represented by three bold lines) and a fragment (represented by two bold lines and a dot) in a ˜3:1 ratio. In salinosporamide B, this labeling pattern was just evident in the cyclohexenyl ring and was completely replaced with a 2+2 unit derived from two acetate molecules in the ethyl substituted γ-lactam.

FIG. 5 is a schematic representation showing a proposed biosynthesis of the salinosporamides via a hybrid PKS-NRPS pathway. Abbreviations: ACP, acyl carrier protein; PCP, peptidyl carrier protein; TE, thioesterase.

FIG. 6 shows the organization and deduced functions of the ORFs in the salinosporamide (sal) biosynthetic gene cluster from Salinispora tropica strains CNB476 and CNB440.

FIG. 7 shows a domain organization of the putative PKS-NRPS proteins SalA and SalB and the proposed modular biosynthesis of intermediates leading to the salinosporamides in S. tropica.

FIG. 8 is a schematic representation showing a proposed biosynthesis of the novel PKS extender unit chloroethylmalonyl-CoA.

FIGS. 9A-9C show results from studies towards the characterization of the chlorinase SalL. FIG. 9A shows the results of purification of SalL after over expression as a fusion protein with an N-terminal His8 tag in E. coli BL21 (DE3). 10% Nu PAGE (Invitrogen). M, molecular weight (MW) marker EZ Run, Fisher Scientific. The molecular weights (kDa) of the marker proteins are indicated. Lane 1, total protein before induction. Lane 2, soluble protein after overnight induction with 0.25 mM IPTG at 20° C. Lane 3, eluate from nickel affinity chromatography. Lane 4, eluate from gel filtration and after thrombin cleavage of the His8 tag. The expected molecular weights of His8-tagged SalL and SalL after thrombin cleavage of the His8 tag are indicated on the right. FIG. 9B shows the results of HPLC analysis of the reaction catalyzed by SalL. The 5′-ClDA standard is shown in red, the negative control (substrates SAM and NaCl incubated without enzyme) in blue and the enzyme containing assay in green (His8-tagged SalL was used). Detection at 254 nm. FIG. 9C is a pictorial diagram showing a crystal (80×60×30 μm) of SalL.

FIG. 10 is a schematic representation showing a proposed biosynthesis of the novel non-proteinogenic amino acid L-3-cyclohexenylalanine via a new shunt in the shikimic acid pathway.

FIGS. 11A-11C show inactivation of SalA in S. tropica. FIG. 11A shows a schematic presentation of the SalA inactivation experiment. The DNA fragment used as probe is indicated as a black bar. FIG. 11B shows the results of southern blot analysis of wild-type and SalA mutants. Genomic DNA was digested with EcoRI. M, DIG DNA molecular weight marker III, Roche. WT, wild-type strain CNB440. 1 and 2, two independent exconjugants. FIG. 11C shows the results of HPLC analysis of secondary metabolites. S. tropica CNB440 wild-type (blue) and salA mutant (red).

FIGS. 12A-12C show a PCR-targeting strategy. FIG. 12A shows the first step is to amplify an antibiotic resistance cassette using primers containing 39-nt 5′ homology extensions (H1, H2) corresponding to regions flanking the target gene. The PCR product is then used to transform an E. coli strain expressing the λ-Red recombination functions and containing the target gene in a genomic library DNA clone, e.g a cosmid. Selection for the antibiotic resistance leads to potential transformants, which are confirmed by PCR and restriction analysis. FIG. 12B. Due to the presence of oriT in the cassette, the targeted DNA clone can be introduced into the actinomycete by conjugation from E. coli ET12567/pUZ8002. The long regions of sequence identity in the inserts promote efficient allelic exchange by homologous recombination, which can be confirmed by PCR and Southern blot analysis. FIG. 12C. If necessary, the resistance cassette can be eliminated using a FLP recombinase expression plasmid in E. coli. This leaves a “scar” sequence of 81 bp. The “scar” DNA clone can then be introduced into the actinomycete mutant by protoplast transformation. Again, homologous recombination will lead to allelic exchange, which can be identified by the loss of the antibiotic resistance as well as by PCR and Southern blot analysis.

FIG. 13 is a schematic representation showing a proposed syntheses of (A) cyclohexenylpyruvate (CP) and cyclohexenylalanine (CA) via cyclohexenylacetaldehyde and (B) L-3-cyclohexenyl-3-hydroxyalanine (L-CHA) via L-serine.

FIG. 14 is a schematic representation showing a proposed synthesis of salinosporamide B-SNAC, -SCoA, and -SPCP substrates for the SalF TE and their bioconversion to the cyclized and/or hydrolyzed salinosporamide B.

FIG. 15 is a schematic representation showing a mutasynthesis of diverse sal inosporamide analogs. See Table 2.

FIGS. 16A-16C show the results from studies towards the characterization of the proteasome β-subunit SalJ. FIG. 16A shows the partial sequence alignment of proteasome β-subunits (minus the N-terminal propeptide that is cleaved off autocatalytically during assembly). Residues highlighted with green are those found within 5 Å of bound salinosporamide A in the yeast proteasome X-ray crystal structure (PDB code 2FAK). The arrow notes the conserved Ala residue at position 49 that is changed to Val in the putative resistance protein SalJ. FIG. 16B shows the X-ray crystal structure of the yeast proteasome (brown) overlaid with a SalJ homology model (blue, constructed with MODELLER129) with covalently-bound salinosporamide A. Residues making up the cyclohexenyl-binding pocket are depicted and labeled. FIG. 16C shows the phylogenetic tree of the S. tropica proteasome β-subunits Stro1610 and the putative resistance protein SalJ with other proteasome β-subunits from eukaryotes, archaea and actinomycetes.

FIG. 17 is pictorial diagram showing an X-ray crystal structure of the homotrimeric chloridase SalL.

FIG. 18 is a graphical diagram showing the results of an HPLC analysis of the reaction catalyzed by ΔSalL. The 5′-ClDA standard is shown in red, the negative control (substrates SAM and NaCl incubated without enzyme) in blue and the enzyme containing assay in green. Detection at 254 nm.

FIG. 19 is a graphical diagram showing 20S proteasome inhibition and resistance by salinosporamide A.

### DETAILED DESCRIPTION OF THE INVENTION

The present invention may be understood more readily by reference to the following detailed description of specific embodiments and the Examples included therein.

Salinosporamide A is a potent anticancer agent that entered phase I human clinical trials in May 2006 for the treatment of multiple myeloma only three years after its discovery from the obligate marine bacterium Salinispora tropica. This novel marine natural product possesses a densely functionalized γ-lactam-β-lactone pharmacophore that is responsible for its irreversible binding to the 20S proteasome, a new drug target validated in cancer biology.

### Chemistry and Biology of the Salinosporamides in Cancer Chemotherapy

Since its introduction in 2003, the potent 20S proteasome inhibitor salinosporamide A1 has rapidly advanced through pre-clinical development and on May 11, 2006, it entered phase I human clinical trials for the treatment of multiple myeloma (Nereus Pharmaceuticals). This novel marine microbial natural product was discovered by William Fenical and co-workers at the Scripps Institution of Oceanography from the newly described marine obligate actinomycete Salinispora tropica isolated from sediments in the Bahamas (Feling et al. 2003 Angew Chem Int Ed 115, 369-371; Mincer et al. 2002 Appl Environ Microbiol 68, 5005-5011; and Madonado et al. 2005 Int J System Appl Microbiol 55, 1759-66).

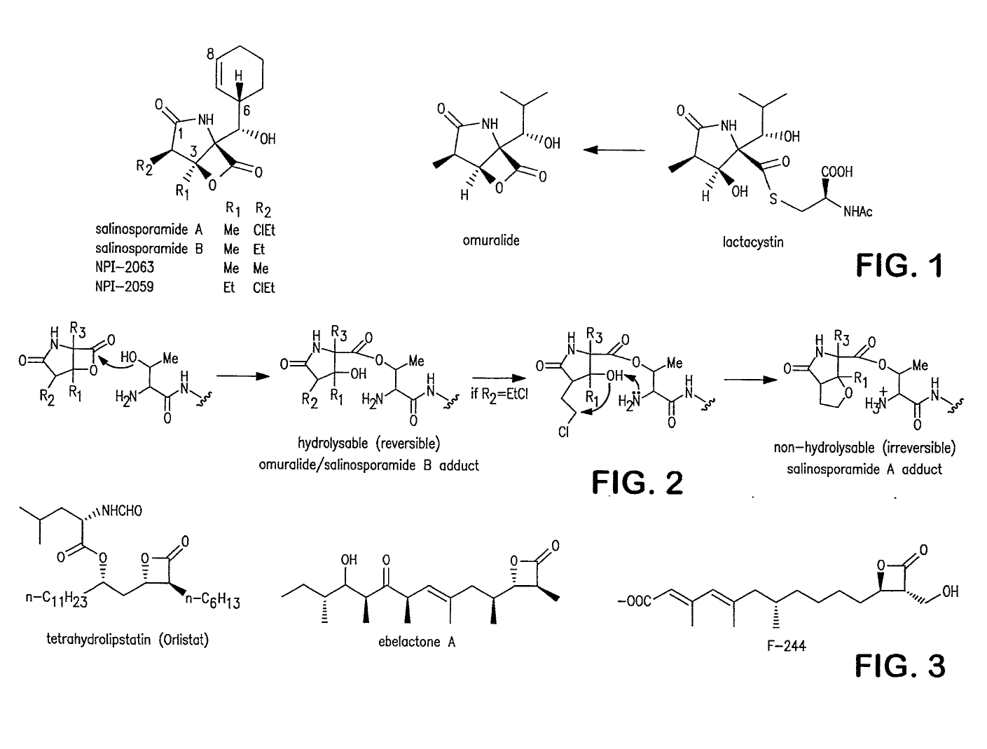

Salinosporamide A is a member of a small family of naturally occurring γ-lactam-β-lactone compounds produced by S. tropica that include different substituents at C-2 and C-3 (FIG. 1). Its rare bicyclic ring system was reported just once before in nature in clasto-lactacystin-β-lactone (also called omuralide7), a transformation product of lactacystin characterized from the terrestrial actinomycete Streptomyces lactacystinaeus in 1991 (FIG. 1). See Omura, et al. 1991 J. Antibiot. 44, 113-1 16; and Omura et al. 1991 J. Antibiot. 44, 117-118.

Extensive studies on lactacystin have since shown that this natural product inhibits the proteasome, a multisubunit protease complex responsible for ubiquitin-mediated degradation, through a covalent interaction with the active site N-terminal nucleophilic threonine residue in the 20S catalytic core. Salinosporamide A has enhanced potency over omuralide (IC50 values of 1.3 nM versus 49 nM, respectively, against purified 20S proteasome), which implicates the different functionalization of the pharmacophore as being mechanistically significant.

Recent crystal structures of the yeast 20S proteasome catalytic core in complex with omuralide (Groll et al. 2004 Biochim. Biophys. Acta 1695, 33-44; and Groll et al. 1997 Nature 386, 463-471), and salinosporamides A and B15 have beautifully illuminated the mechanism of this family of β-lactone inhibitors in which the drug is linked through an ester bond to the side chain hydroxyl group of the N-terminal threonine residue. In the case of salinosporamide A, however, further chemistry ensues in which the newly created C-3 hydroxyl adds to the C-2 chloroethyl group to give a cyclic ether (FIG. 2). This intramolecular nucleophilic addition is unique to salinosporamide A and was first observed with the natural product in aqueous solution. The consequence of the formation of the tetrahydrofuran ring is that it renders the ligand irreversibly bound to the enzyme, as the C-3 oxygen atom occupies the same position as that for the hydrolytic water molecule in the unligated enzyme, thereby hindering deacylation. The unreactive C-2 methyl and ethyl groups in omuralide and salinosporamide B, respectively, prevent the subsequent formation of such a ring and results in a slowly reversible enzyme ligand complex. It was recently reported that full recovery of blood 20S proteasome activity was restored within 24 hours upon treatment with a synthetic analogue of omuralide, whereas it was only partially restored in mice after 7 days upon treatment with salinosporamide A (possibly due to red blood cell turnover). In lieu of the chloro leaving group, the C-3 hydroxyl group in the ligated omuralide and salinosporamide B complexes may either reform the β-lactone ring or alternatively allow a water molecule to bind for hydrolysis. Hence depending on the reactivity of the C-2 substituent, nature has evolved potent reversible and irreversible inhibitors of the proteasome around the γ-lactam-β-lactone nucleus.

Still, other 20S proteasome inhibitors besides salinosporamide A (aka NP1-0052) are also encompassed by the present invention including but not limited to, lactacystin, Ac-PRLN-vs, ajoene, Acetyl-Leu-Leu-NorLeucinal (Ac-LLN-al), Gold(III) dithiocarbonate, bortezomib, PS-341, PS-519 and MG-132.

### The 20S Proteasome as a Validated Target for the Treatment of Cancer

The discovery of the mechanism of action of lactacystin/omuralide helped clarify the mechanistic enzymology behind the proteasome as a novel threonine protease and led the way to the characterization of the importance of the ubiquitin-proteasome pathway in a number of key cellular processes. Since the discovery of lactacystin, the proteasome has rapidly emerged as a new therapeutic target for a number of human diseases, including cancer, inflammation, and even anthrax infections. See Kisselev et al. 2001 Chem. Biol. 8, 739-758; Ciechanover, A. (2006). Neurology 66 Suppl. 1, S7-S19; Voorhee et al. 2003 Clin. Cancer Res. 9, 6316-6325; Kaufman, et al. 2006 Onkologie 29, 162-168; Bazzaro et al. 2006 Cancer Res. 66, 3754-3763; Smith et al. 2006 Lett. Drug Design Discov. 2, 74-81, Elliott et al. 2003 J. Mol. Med. 81, 235-245; and Tang et al. 1999 Infect. Immun. 67,3055-3060.

With the FDA approval of the synthetic peptide boronate bortezomib (Velcade, PS-341; Millennium Pharmaceuticals) in 2003 for the treatment of multiple myeloma, the 20S proteasome has become a validated target for cancer chemotherapy. Other peptide-derived classes of proteasome inhibitors, including peptide aldehydes, peptide vinyl sulfones, and peptide epoxyketones, have been synthetically designed and hold promise as specific inhibitors as well.

The non-peptide based inhibitor salinosporamide A (aka NPI-0052) is chemically and mechanistically distinct from the peptide-based inhibitors such as bortezomib and represents an alternative in multiple myeloma therapy. Moreover, salinosporamide A reportedly induces apoptosis in multiple myeloma cells that have become refractory to bortezomib therapy. Recently, researchers have reported that salinosporamide A is orally active, well tolerated, and prolongs survival in animal tumor models. In addition to its activity against multiple myeloma, salinosporamide A is efficacious in animal models against colon, pancreatic, and lung cancers.

### Natural Product/Lactones as Drugs

The chemical reactivity of the electrophilic β-lactone group in omuralide and the salinosporamides is reminiscent to that of the β-lactam antibiotics, which target serine transpeptidases in bacterial cell wall biosynthesis. While the chemistry and pharmacology of β-lactam antibiotics such as penicillins, cephalosporins, β-lactamase inhibitors, carbapenems, and monobactams has a rich history and is firmly entrenched in chemistry and medical textbooks, β-tactone-containing natural products, although rare, are starting to gain their own notoriety. In addition to omuralide and the salinosporamides, which contain a syn β-lactone group, bacteria also produce a variety of lipids that possess reactive β-lactone groups with an anti relative configuration (FIG. 3). Lipstatin, a product of Streptomyces toxytricini, is a potent and irreversible inhibitor of pancreatic lipase, and belongs to a family of structurally related β-lactones from actinomycetes that include the ebelactones, panclicins, esterastin, and valilactone. Tetrahydrolipstatin, which is also known as Orlistat and is marketed as Xenical, was approved by the FDA in 1999 for weight management in obese patients. See Heck et al. 2000 Pharmacother. 20, 270-279; and Hollander, P. 2003 Prim. Care 30, 427-440. Inhibition of the pancreatic (as well as other) lipase by the covalent modification of the active site serine residue limits the absorption of dietary fat. Tetrahydrolipstatin also inhibits the thioesterase (TE) domain in fatty acid synthase (FAS) in tumor cells by the covalent attachment to its active site serine residue and is being pursued as a potential antitumor therapeutic.

The isoprenoid biosynthetic enzyme 3-hydroxy-3-methylglutaryl-CoA synthase (HMGS), which catalyzes the condensation of acetoacetyl-CoA and acetyl-CoA, is also specifically inhibited by a natural anti-configured β-lactone. The fungal polyketide F-244 (hymegiusin, L-659,699) (FIG. 3) is a potent inhibitor of HMGS with an IC50 of 35 nM and covalently binds to the active site cysteine residue of this α-keto-thiolase folded protein. Its selective inhibition of HMGS has promise as a cholesterol-lowering agent much like the statins that target HMG-CoA reductase.

### Biosynthesis of β-Lactone Natural Products

Besides feeding experiments with radio and stable isotopes, very little is known about how β-lactone natural products are biosynthesized or how the producing organism resists these protein-reactive compounds. In the case of lipstatin, extensive labelling studies have shown that the β-lactone unit is formed by the condensation of C14 and C8 fatty acid residues. For lactacystin, which is more structurally relevant to the salinosporamides, feeding experiments with basic precursors established the origin of the carbons as L-leucine, L-cysteine and isobutyrate (via L-valine). The γ-lactam moiety is proposed to form via a condensation between methylmalonic semialdehyde and the α-carbon of leucine followed by an intramolecular condensation.

The enzymology behind β-lactone formation in nature has not been established. Insight may be taken from characterized β-lactam forming enzymes isopenicillin N synthase, a non-heme iron-dependent oxidase involved in penicillin and cephalosporin biosynthesis and β-lactam synthetase, an ATP/Mg2+-dependent enzyme involved in the synthesis of monobactams and the β-lactamase inhibitor clavulanic acid. The latter example seems mechanistically plausible for the formation of a β-lactone group by the activation of the carboxylate with ATP to the AMP derivative to facilitate β-lactone formation through an intramolecular displacement of AMP by the β-hydroxy group.

Preliminary studies on the biosynthesis of salinosporamide A were presented at the 45^{th} Annual Meeting of the American Society of Pharmacognosy, Phoenix, Ariz., July 2004, and at the Genetics and Microbiology of Industrial Microorganisms/Biotechnology of Microbial Products Conference, San Diego, Calif., November 2004. The poster presentation was titled “Proposed biosynthesis of the marine natural product salinosporamide A: a novel 20S proteasome inhibitor”; and the contents of which are incorporated herein in their entirety by reference.

In one embodiment of the present invention, there is provided a method for isolation, cloning, sequencing and characterizing the salinosporamide biosynthetic gene cluster of S. tropical strains CNB440. The complete nucleotide sequence of the biosynthetic gene cluster allows continued and further studies to chemically and genetically re-engineer the biosynthetic pathway to generate new salinospora analogs for various research and therapeutic uses, for example: biological testing; improve production yields of the drug salinosporamide A, e.g., through fermentation, or other methods known in the art, with precursor intermediates; reduce and/or eliminate in substantial or minor, natural analogs through re-engineering of the pathway (e.g., mutagenesis and/or mutasynthesis) to decrease or eliminate natural structural analogs or decontaminants to simplify the large-scale purification of salinosporamide A; and generate unnatural or not naturally occurring analogs to salinosporamide A with different biological and physical properties.

As used herein, the terms “yield,” “chemical yield” and “reaction yield,” refer to the amount of product obtained in a chemical reaction. The term “absolute yield” refers to the weight in grams or in moles (molar yield). The terms “percentage yield” or “relative yield,” refer to the effectiveness of a synthetic procedure, which is calculated by dividing the amount of the obtained product in moles by the theoretical yield in moles. Yields may appear to be above 100% when products are impure. Purification steps always lower the yield and the reported yields usually refer to the yield of the final purified product.

Currently, the salinosporamides A is produced in fermentation by the bacterium Salinispora tropica. Thus, the present invention provides for the first time methods of biochemical and genetic characterization of the salinosporamide a biosynthetic pathway in S. tropics. This information provides a means to increase the production yield and purity of the promising anti-cancer agent salinosporamide a through fermentation. The present invention by providing methods to make biosynthetic salinosporamide A and analogs thereof establishes the biological building blocks of this product by identifying the putative set of genes involved in its regulation, resistance and biosynthesis. For example, at present, the analog salinosporamide B is a fermentation artifact which makes up about 1-5% of the total salinosporamides yield. The present invention demonstrates that the structural difference between salinosporamides A and B, which have chlorethyl and ethyl residues, respectively, is reflected in biosynthesis involving an erythrose-derived molecule and butyrate, respectively. Hence, the discovery and validation of the salinosporamide biosynthetic gene cluster, provides the molecular basis for the biosynthesis and attachment of each building block precursor.

In another embodiment of the invention, there is provided a method to produce salinosporamide analogs (natural and not naturally occurring) in fermentation with genetically engineered bacterial strains. This approach has many possible advantages over the current production method employing the wild type strain. The present invention provides a means to generate novel analogs for biological evaluation, for example, a screening assay, as well as to increase production yields while eliminating nuisance compounds through genetic engineering.

For example, the present invention provides for the first time a method to introduce DNA into the described bacterial genus for the inactivation of genes. Inactivation of the gene salL, which encodes a novel 5′-chloro-5′-deoxyadenosine synthase, through PCR targeting provided a mutant S. tropica strain devoid exclusively of salinosporamide a biosynthesis. Chemical complementation of this mutant with ClDA restored salinosporamide a biosynthesis and validated the selective targeting of the C-2 side chain through mutagenesis.

Inactivation of the gene salD, which encodes a cytochrome P450, through PCR targeting provides a mutant S. tropica strain devoid of all 5-hydroxy salinosporamide biosynthesis, and provides a means to exclusively produce 5-deoxy derivatives.

Inactivation of the gene salg, which encodes a crotonyl-coenzyme A reductase, through PCR targeting provided a mutant S. tropica strain devoid exclusively of salinosporamide A biosynthesis and like the ΔsalL mutant provides a means to generate greater titers of the natural minor salinosporamides.

The present invention provides methods for determining the biosynthetic building blocks of salinosporamide A and analogs thereof by feeding experiments with stable isotopes (e.g., ¹³C), other detectable isotopes and/or labels are also embodied in the invention for example, the detectable marker is a radioactive isotope, enzyme, dye, biotin, fluorescent label or chemiluminescent label.

Characterization of salinosporamide A gene cluster as described herein, can also be used to manufacture and produced high throughput assays, e.g., high throughput immunoassays, high throughput screening assays to identify and detect various agents which effect the biosynthesis of the salinosporamide A or an analog thereof, high throughput detection assays to determine agents, peptides, polypeptides, small molecules, toxins, and variants thereof which bind to or inhibit binding of salinosporamide A and/or its analog thereof, and high throughput assays which identify and detect agents, peptides, polypeptides, small molecules, toxins, and variants thereof which are activated and/or inhibited by salinosporamide A and/or its analog. These and other aspects of the invention are provided by or anticipated by the present invention described herein.

The elucidation of the biosynthetic pathway at the chemical, biochemical and genetic level to the salinosporamides will provide a number of opportunities to impact how salinosporamide A is commercially produced in the long-term and to afford ready access to new fermentation-based chemical variants for SAR studies through rational metabolic engineering. In addition, its unique chemical structure provides a number of rare opportunities to discover new biosynthetic processes that may have applied value as biocatalysts. Also, to date, there are no reports on how this fermented drug is naturally created. Salinosporamide A is currently produced through fermentation of the bacterium S. tropica. The present invention provides an improved alternative means to generate salinosporamide A and analogs for biological evaluation as well as to increase production yields while eliminating nuisance compounds through genetic engineering.

In one embodiment of the invention, there is provided a method of deriving non-naturally occurring salinosporamide analogs. As used herein, the term “analog,” “analogues” or grammatical equivalents thereof, refers to an analog having a component, for example, element, an atom, a bond, an amino acid sequence or a nucleotide sequence that is not naturally occurring. For example, analogs are compounds in which one or more individual atoms have been replaced, either with a different atom, or with a different functional group.

### Antibodies to Polypeptide of the Sal Gene Cluster Including Salinosporamides A and Analogs Thereof

In another aspect of the invention, antibodies are provided which bind to any of the biosynthetic components of salinosporamide A and/or an analog thereof. These antibodies may also be attached to solid supports, e.g., antibodies are particularly useful for immunoassays or immunoprecipitation of any of the polypeptides and/or biosynthetic enzymes described herein. Such solid supports include, but are not limited to, glass, cellulose, polyacrylamide, nylon, polystyrene, polyvinyl chloride or polypropylene, for example protein G covered wells of microtiter plates or beads.

Antibodies directed against a specific epitope, or combination of epitopes, so as to bind specifically with any of the biosynthetic components of salinosporamide A and/or its analog thereof will allow for the screening of, for example, cell populations as described herein. Various screening techniques can be utilized using such polyclonal and monoclonal antibodies, and including magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

The antibodies useful in the invention methods may be assayed for immunospecific binding by any method known in the art. The immunoassays which can be used, include but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., Western blot analysis. Those of skill in the art would be knowledgeable as to the parameters that can be modified to increase the binding of the antibody to an antigen and decrease the background (e.g., pre-clearing the cell lysate with sepharose beads.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. Those of skill in the art would be knowledgeable as to the parameters that can be modified to increase the signal detected and to reduce the background noise.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. Those of skill in the art would be knowledgeable as to the parameters that can be modified to increase the signal detected as well as other variations of ELISAs known in the art.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with the antibody of interest conjugated to a labeled compound (e.g., ³H or ¹²⁵I) in the presence of increasing amounts of an unlabeled second antibody.

Antibodies used in invention assay(s) can be polyclonal, monoclonal, or a functionally active fragment thereof. Mono- or poly-clonal antibodies to a salinosporamide A are raised in appropriate host animals by immunization with immunogenic conjugate(s) using conventional techniques as are known in the art.

The preparation of monoclonal antibodies is disclosed, for example, by Kohler and Milstein, Nature 256:495-7, 1975; and Harlow et al., in: Antibodies: a Laboratory Manual, page 726 (Cold Spring Harbor Pub., 1988), which are hereby incorporated by reference. Briefly, monoclonal antibodies can be obtained by injecting mice, or other small mammals, such as rabbits, with a composition comprising an invention immunogenic conjugate whose preparation is disclosed above, verifying the presence of antibody production by removing a serum sample, removing the spleen to obtain B lymphocytes, fusing the B lymphocytes with rnyeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Barnes et al., Purification of Immunoglobulin G (IgG), in: Methods in Mol. Biol., 10: 79-104,1992). Antibodies of the present invention may also be derived from subhuman primate antibodies.

It is also possible to use anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

The term “antibody” as used in this invention includes intact molecules as well as functional fragments thereof, such as Fab, F(ab′)₂, and Fv that are capable of binding salinosporamide A polypeptide These functional antibody fragments are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule that can be obtained by treating whole antibody with pepsin; followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference). As used in this invention, the term “epitope” means any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

Antibody fragments according to the present invention can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells.

The invention methods use monoclonal antibodies characterized as specifically binding to any biosynthetic component(s) of the salinosporamide A or B and their analogs thereof.

Hybridoma cell lines producing monoclonal antibodies useful in the invention methods for immunocapture of any biosynthetic component(s) of the salinosporamide A or B and their analogs thereof can be made commercially available.

### Biosynthesis of the Novel PKS Extender Unit Chloroethylmalonyl-CoA

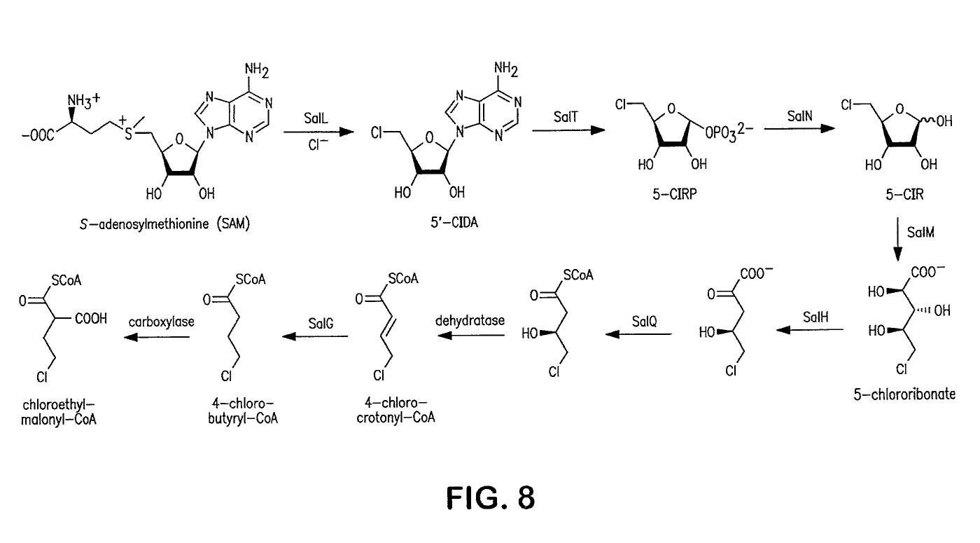

Based on preliminary data, it was hypothesized that the chlorinated tetrose-derived unit in salinosporamide A originates via a new pathway in which SAM is converted in nine steps to the novel PKS extender unit chloroethylmalonyl-CoA (FIG. 8). To elucidate the pathway to this chlorinated precursor, a tandem approach involving in vivo and in vitro techniques was employed. These studies will provide the opportunity to bio-engineer new structural units at C-2 (R2 group) and bio-engineer other halogenated polyketides.

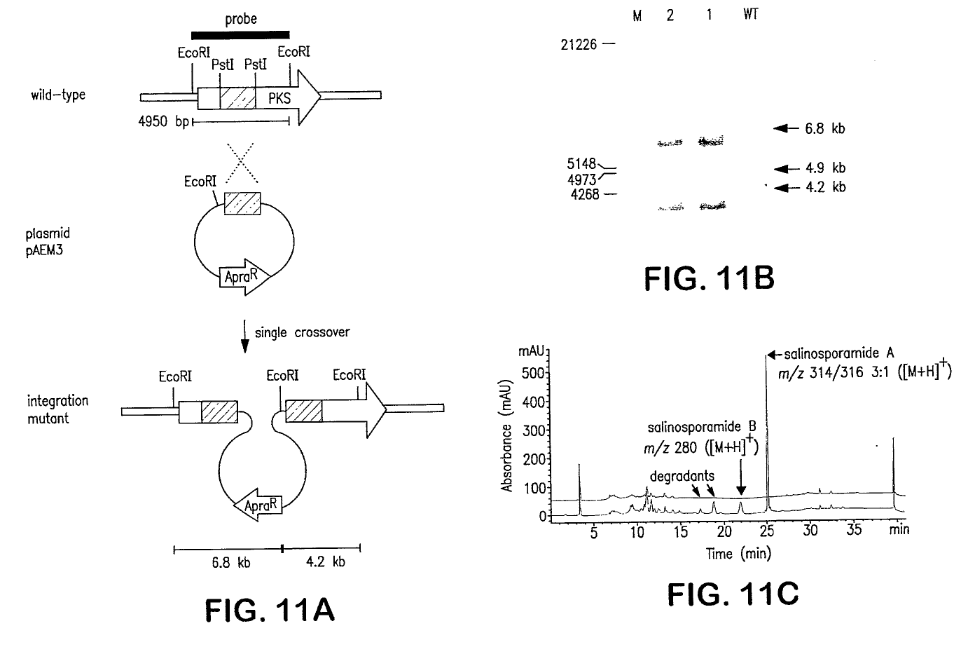

In one embodiment of the present invention, there is provided various in vivo assays and methods. For example, inactivation of the PKS gene salA via a classical approach involving the single crossover homologous recombination of the suicide vector pAEM3 (FIG. 11). However, this procedure for gene deletions via double crossover homologous recombination to stable mutants is time consuming. Hence, a PCR-based mutagenesis approach for S. coelicolor is being adapted for actinomycetes. This approach has been successfully employed on novobiocin and chlorobiocin, and is being adapted to S. tropica. See Eustáquio et al. 2005 Appl. Environ. Microbiol. 71, 2452-2459; Eustáquio et al. 2004 Chem. Biol. 11, 1561-1572; Eustáquio et al. 2003 Chem. Biol. 10, 279-288; and Eustáquio et al. 2003 Arch. Microbiol. 180, 25-32. Additional PCR-targeting mutagenesis can be performed using information from a 40-kb genomic library and a REDIRECT© technology kit containing several E. coli strains and antibiotic resistance cassettes.

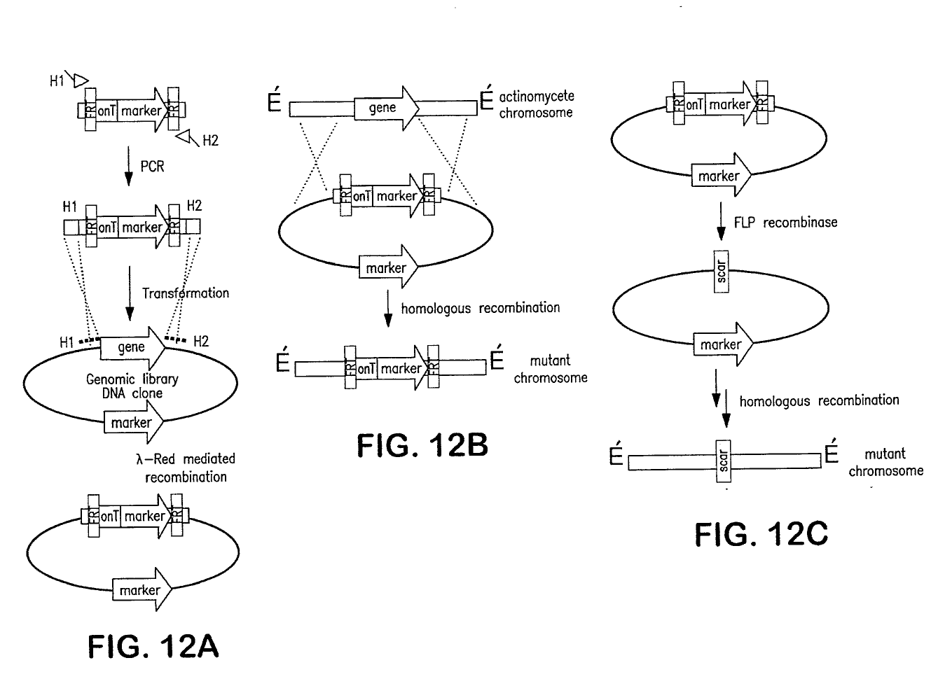

The strategy for PCR-targeting for mutagenesis of actinomycetes (FIG. 12) is to replace a chromosomal sequence within a library clone (e.g. a cosmid) by a selectable marker that has been generated by PCR using primers with 39 nt homology extensions. Recombination of these short homologous sequences with the cosmid DNA contained in E. coli is promoted by the λ Red functions (gam, bet, exo). Gam inhibits E. coli exonucleases, which would otherwise degrade linear DNA, while Bet and Exo promote recombination. The inclusion of oriT in the disruption cassette allows conjugation to be used to introduce the targeted cosmid DNA into the desired actinomycete. Once in the actinomycete, the vector used for the library construction does not replicate autonomously, but the long regions of sequence identity in the inserts promote efficient integration by homologous recombination, thus creating gene replacements. Indeed over about 10% of the obtained mutants typically undergo double crossover events, meaning that the desired gene replacement can be obtained directly after conjugation making time consuming screenings unnecessary. Furthermore, the antibiotic resistance cassettes are flanked by yeast FLP recombinase target sequences (FRT) for removal of the cassette to generate unmarked, nonpolar mutations if necessary. Additionally, standard molecular biology techniques employing deletion experiments by double crossover homologous recombination using a similar approach to that successfully employed in the inactivation of salA (FIG. 11 is anticipated.

For example, a knockout of the pathway specific for the chlorinase gene salL would result in the loss of salinosporamide A, its degradation products and NPI-2059. This mutant should result in the (over) production of salinosporamide B (butyrate extender unit), NPI-2063 (propionate unit) and possibly other natural analogs previously missed due to the over abundant production of salinosporamide A. Further, the mutant can be complemented or supplemented chemically with 5′-ClDA (commercially available from Aldrich) and genetically with the wild-type salL gene in an appropriate vector to restore activity as a further proof of salL gene function. An appropriate vector for the genetic complementation experiments, for example, can be the E. coli streptomycete expression vector pBM6 containing the strong constitutive ermE* promoter with oriT to allow conjugation from E. coli. Organic extracts of the mutant and complemented mutant will be characterized by HPLC-MS with the aid of salinosporamide standards as routinely employed in the laboratory.

Mutagenesis and/or knockout studies of the various biosynthetic pathway enzymes can be used to elucidate protein function. For example, various genes in the cluster can inactivated and then chemically and/or genetically complemented. Inactivation of at least chlorobutyryl-CoA biosynthesis genes may result in the complete abolishment of salinosporamide A biosynthesis as some genes may be partially complemented by primary metabolic homologs (Table 1). In cases where salinosporamide A is retained, relative production by HPLC against salinosporamide B and/or NPI-2063 can be measured. One gene of particular interest is the crotonyl-CoA reductase (CCR) homolog salG, which likely functions primarily as a chlorocrotonyl-CoA reductase that catalyzes the penultimate reaction in chloroethylmalonyl-CoA synthesis (FIG. 8). On the other hand, it may be a bona fide CCR involved in the synthesis of the salinosporamide B extender unit ethylmalonyl-CoA (as is the case with other butyrate containing PKS gene clusters) and have less to do with salinosporamide A biosynthesis. HPLC analysis of the salG mutant should be able to differentiate between these two scenarios as either salinosporamide A or B should be reduced or possibly abolished while production of the propionate extended analogs NPI-2063 should remain unchanged (or slightly increase). Depending on the outcome, fermentation and growth can be complemented by feeding 4-chlorobutyric acid and/or butyric acid to rescue salinosporamide A or B synthesis, respectively. Alternatively, the cultures may be fed corresponding N-acetylcysteamine (NAC) thioester analogs. Such gene knockout experiments coupled with corresponding in vitro studies (below) will allow refinement of this novel pathway.

TABLE 1
Comparison of proposed sal chloroethylmalonyl-CoA pathway genes versus genomic homologs

| sal gene | proposed function | stro gene | proposed function | % identity |
|---|---|---|---|---|
| salL | 5′-ClDA synthase (chlorinase) | none | | |
| salT | 5′-ClDA phosphorylase | none | | |
| salN | 5-ClRP phosphatase | none | | |
| salM | 5-ClR 1-dehydrogenase | stro2290 | short-chain dehydrogenase | 36 |
| salH | 5-chlororibonate dehydratase | stro4465 | dihydroxyacid dehydratase | 36 |
| salQ | α-ketoacid decarboxylase | stro307 | pyruvate decarboxylase | 94 |
| none | 4-Cl-3-OH-butyryl-CoA dehydratase | | 3-hydroxybutyryl-CoA dehydratase¹ | |
| salG | (chloro)crotonyl-CoA reductase | stro2345 | CCR | 53 |
| none | chlorobutyryl-CoA-carboxylase | | acyl-CoA carboxylase² | |

¹There are ~12 candidate dehydratases present in the genome.
²There are 4 candidate carboxylases in the genome.

In another embodiment of the invention, there is provide in vitro assays and methods. For example, various genes in the cluster can be inserted into an expression vector system, e.g., E. coli expression plasmids, pET-28a(+) (Novagen) and a derivative of this plasmid, pHIS8. A recombinant expression vector generally refers to a plasmid, virus or other vehicle known in the art that has been manipulated by insertion or incorporation of a nucleic acid sequences. For example, a recombinant expression vector of the invention includes a polynucleotide sequence encoding a SalA analog polypeptide or a fragment thereof. The expression vector typically contains an origin of replication, a promoter, as well as specific genes which allow phenotypic selection of the transformed cells. Vectors suitable for use in the invention include, but are not limited to the T7-based expression vector for expression in bacteria, the pMSXND expression vector for expression in mammalian cells, baculovirus-derived vectors for expression in insect cells, cauliflower mosaic virus, CaMV; tobacco mosaic virus, TMV.

Both pET-28a(+) and pHIS8 plasmids encode an N-terminal histidine tag followed by a thrombin cleavage site for removal of the polyhistidyl-containing segment from expressed proteins. The fidelity of the PCR products will be confirmed by automated nucleotide sequencing. Expression can be carried out in E. coli BL21 (DE3) and the protein purified using various methods known in the art, including: a His-tag purification system on Ni2+-NTA column, thrombin cleavage and removal with a benzamidine-Sepharose column, anion-exchange purification on a Mono Q column and/or gel filtration on a Superdex-200 column. Alternatively, E. coli—Streptomyces shuttle expression vector pXY200 for the production of His6-tagged proteins in Streptomyces lividans TK24 can also be employed. In fact, Streptomyces is more closely related to S. tropica. This alternative approach has been used in the past to express the enterocin minimal PKS.

In the present invention, nucleotide sequences encoding SalA and/or analog and/or functional fragments thereof, are inserted or incorporated into E.coli vectors. However, other vectors from other systems can also be used. For example, in yeast, a number of vectors containing constitutive or inducible promoters may be used. Alternatively, vectors may be used which promote integration of foreign DNA sequences into the yeast chromosome.

Nucleotide sequences of the invention may also be inserted into an expression system which expresses an agent which modulates expression or activity of SalA and/or analog or fusion or functional fragments thereof in an insect system (e.g., Drosophilia). In one such system, Autographa californica nuclear polyhedrosis virus (AcNPV) is used as a vector to express foreign or mutated polynucleotide sequences. The virus grows in Spodoptera frugiperda cells. The sequence encoding a protein of the invention may be cloned into non-essential regions (for example, the polyhedrin gene) of the virus and placed under control of an AcNPV promoter (for example the polyhedrin promoter). Successful insertion of the sequences coding for a protein of the invention will result in inactivation of the polyhedrin gene and production of non-occluded recombinant virus (i.e., virus lacking the proteinaceous coat coded for by the polyhedrin gene). These recombinant viruses are then used to infect S. frugiperda cells in which the inserted gene is expressed.

The vectors of the invention can be used to transform a host cell, e.g., a prokaryotic or eukaryotic host cell. As used herein, the term “transform” or “transformation” or equivalents thereof, refers to a permanent or transient genetic change induced in a cell following incorporation of new DNA (i.e., DNA exogenous to the cell). Where the cell is a mammalian cell, a permanent genetic change is generally achieved by introduction of the DNA into the genome of the cell.

As used herein, a “transformed cell” or “host cell” generally refers to a cell (e.g., prokaryotic or eukaryotic) into which (or into an ancestor of which) has been introduced, by means of recombinant DNA techniques, a DNA molecule encoding an agent which modulates expression or activity of salinosporamide A and or analog or functional fragment thereof.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, methods of transfection or transformation with DNA include calcium phosphate co-precipitates, conventional mechanical procedures such as microinjection, electroporation, insertion of a plasmid encased in liposomes, or virus vectors, as well as others known in the art, may be used. Eukaryotic cells can also be co-transfected with DNA sequences encoding an agent which modulates expression or activity of SalA and/or analog or fusion or functional fragments thereof and a second foreign DNA molecule encoding SalA and/or analog, or a selectable marker, such as the herpes simplex thymidine kinase gene. Another method is to use a eukaryotic viral vector, such as simian virus 40 (SV40) or bovine papilloma virus, to transiently infect or transform eukaryotic cells and express the protein. Typically, a eukaryotic host will be utilized as the host cell. The eukaryotic cell may be a yeast cell (e.g., Saccharomyces cerevisiae), an insect cell (e;g., Drosophila sp.) or may be a mammalian cell, including a human cell.

Eukaryotic systems, and mammalian expression systems, allow for post-translational modifications of expressed mammalian proteins to occur. Eukaryotic cells which possess the cellular machinery for processing of the primary transcript, glycosylation, phosphorylation, and, advantageously secretion of the gene product should be used. Such host cell lines may include, but are not limited to, CHO, VERO, BHK, HeLa, COS, MDCK, Jurkat, HEK-293, and WI38.

Mammalian cell systems which utilize recombinant viruses or viral elements to direct expression may be engineered. For example, when using adenovirus expression vectors, a polynucleotide encoding an agent which modulates expression or activity of salinosporamide A or analog, or salinosporamide A or analog fusion, or salinosporamide A or analog functional fragments thereof may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a encoding an agent which modulates expression or activity of SalA and/or analog or fusion, or functional fragments thereof in infected hosts. Alternatively, the vaccinia virus 7.5K promoter may be used. Of particular interest are vectors based on bovine papilloma virus which have the ability to replicate as extra chromosomal elements. Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression a gene encoding an agent which modulates expression or activity of synGAP or synGAP fusion or synGAP functional fragments thereof gene in host cells. High level expression may also be achieved using inducible promoters, including, but not limited to, the metallothionine IIA promoter and heat shock promoters.

For long-term, high-yield production of recombinant proteins, stable expression may be required. Rather than using expression vectors which contain viral origins of replication, host cells can be transformed with the cDNA encoding an agent which modulates expression or activity of activity of SalA and/or analog or fusion, or functional fragments thereof controlled by appropriate expression control elements (e.g., promoter, enhancer, sequences, transcription terminators, polyadenylation sites, and the like), and a selectable marker. The selectable marker in the recombinant vector confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used, including, but not limited to, the herpes simplex virus thymidine kinase, hypoxanthine-guanine phosphoribosyltransferase, and adenine phosphoribosyltransferase genes can be employed in tk-, hgprt- or aprt-cells respectively. Also, anti-metabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin genes. Recently, additional selectable genes have been described, namely trpB, which allows cells to utilize indole in place of tryptophan; hisD, which allows cells to utilize histinol in place of histidine; and ODC (ornithine decarboxylase) which confers resistance to the ornithine decarboxylase inhibitor, 2-(difluoromethyl)-DL-ornithine, DFMO.

In order to amplify agents of the invention that are nucleic acid molecules, methods of amplifying such molecules are standard and well known in the art, including those that use of nucleic acid primers and the like. As used herein, the term “primer” refers to an oligonucleotide, whether natural or synthetic, which is capable of acting as a point of initiation of synthesis when placed under conditions in which primer extension is initiated or possible. Synthesis of a primer extension product which is complementary to a nucleic acid strand is initiated in the presence of nucleoside triphosphates and a polymerase in an appropriate buffer at a suitable temperature. For instance, if a nucleic acid sequence is inferred from a protein sequence, a primer generated to synthesize nucleic acid sequence encoding the protein sequence is actually a collection of primer oligonucleotides containing sequences representing all possible codon variations based on the degeneracy of the genetic code. One or more of the primers in this collection will be homologous with the end of the target sequence. Likewise, if a “conserved” region shows significant levels of polymorphism in a population, mixtures of primers can be prepared that will amplify adjacent sequences.

The construction of expression vectors and the expression of genes in transfected cells involves the use of molecular cloning techniques also well known in the art. (See, for example, Sambrook et al., Molecular Cloning—A Laboratory Manual, 3rd Ed., Cold Spring Harbor Laboratory, Cold Spring Harbor, N.Y., 2001, and Current Protocols in Molecular Biology, M. Ausubel et al., eds., (Current Protocols, a joint venture between Greene Publishing Associates, Inc. and John Wiley & Sons, Inc., most recent Supplement)). These methods include in vitro recombinant DNA techniques, synthetic techniques and in vivo recombination/genetic recombination. (See also, Maniatis, et al., Molecular Cloning A Laboratory Manual, Cold Spring Harbor Laboratory, N.Y., 1989).

To determine the steady state kinetic parameters for SalL, HPLC-based assay similar to that employed with the related fluorinase of S. cattleya will be utilized. See Schaffrath et al. 2003 FEBS Lett. 547, 111-114. The SalL product 5′-ClDA (Aldrich) will be used as a standard when testing the other halides for reactivity. While the fluorinase reportedly does not have brominating activity, SalL is likely to as the in vivo addition of bromide to the culture medium amazingly results in the production of the brominated analog of salinosporamide A. Thus not only would SalL have to deliver bromide to SAM to generate 5′-BrDA, but the brominated product would have to be further converted in a series of enzymatic reactions to the natural product. To determine this enzyme's preference for chloride (and presumably bromide) over fluoride, the SalL crystal structure is being solved by molecular replacement using MOLREP94. The X-ray structure of fluorinase showed that Ser158 contributes an important hydrogen bonding contact to fluoride. See Dong et al. 2004 Nature 427, 561-565; and Senn et al. 2005 J. Am. Chem. Soc. 127, 13643-13655. This residue is replaced with glycine in SalL, which may partially explain its preference for the softer chloride ion. To test this idea, the fluorinase-equivalent SalL G131S mutant will be generated by site-directed mutagenesis using the QuikChange (Stratagene) PCR method and test for halide specificity. Once the SalL protein structure has been solved and compared with fluorinase, other mutagenesis experiments may become apparent to understand halide preference in these enzymes. Halide specificity can be switched and then the mutant nucleic acid can be cloned into pWHM3-based expression vector pBM6 to introduce it into the SalL knockout mutant by conjugation for the production of salinosporamide A halogenated analogs.

In another aspect of the invention, other chloroethylmalonyl-CoA pathway enzymes will be characterized, including but not limited to, 5′-CIRP phosphorylase SalT, the dehydrogenase SalM, and the crotonyl-CoA reductase (CCR) SalG. These enzymes putatively catalyze key reactions in the pathway. After the chlorinase, the next step in the putative pathway is catalyzed by the purine nucleoside phosphorylase SalT in an analogous manner to that in the fluoroacetaldehyde pathway in which 5′-XDA is converted to 5-halo-5-deoxy-D-ribose-1-phosphate (5-XRP) and adenine (FIG. 8). It is at this point that the pathways appears to diverge. In the fluoroacetaldehyde pathway, 5-FRP is suggested to undergo an isomerization reaction to 5-fluoro-5-deoxy-D-ribulose-1-phosphate, while in the putative sal chloroethylmalonyl-CoA pathway, 5-CIRP is dephosphorylated to 5-chloro-5-deoxy-D-ribose (5-CIR) by the phosphatase SalN. The gene products SalT and SalN do not have homologs in the sequenced S. tropica genome (Table 1). SalN has been cloned into pHIS8 and the recombinant protein over expressed in substantially the same way as was described for SalL. Again, this enzyme will be assayed using the substrate 5-CIRP prepared from a chemoenzymatic method, which involves the coupling of the chlorinase SalL and a purine nucleoside phosphorylase (commercial or even recombinant SalT). The enzymatically prepared 5-CIRP in the presence of recombinant SalN will be assayed directly by ESI-MS or indirectly using a colorimetric technique, which is used for determining the presence of inorganic phosphate.

Chlororibose (5-CIR) is then oxidized by the dehydrogenase SalM, which is homologous to ribose 1-dehydrogenase (NADP+), to give the trihydroxyacid product chlororibonate (FIG. 8). SalM has been cloned and expressed in E.coli BL21(DE3) and yielded soluble protein. Again, this enzyme will be assayed using the substrate 5-CIR prepared via an enzymatic method from 5′-ClDA and a nucleosidase substantially as described above; 5′-ClDA and a nucleosidase are both commercially available. Alternatively, a synthetic sample of 5-CIR can be prepared in three steps from ribose according to the literature. See, Gudmundsson et al. 1997 J. Med. Chem. 40, 785-793. The enzymatic reaction will monitor the reduction of the cofactor (NAD or NADP) in a similar manner to that outlined for SalG below. Thus, with SalM over expressed, other substrate analogues for SalM can be studied and characterized to determine whether the enzyme has a preference for this chlorinated substrate. For example, SalM can be prepared to its primary homolog Stro2290 (Table 1), which based on sequence analysis, belongs to the well known short-chain dehydrogenase/reductase family (SDR).

In another aspect of the invention, the crotonyl-CoA reductase (CCR) SalG will be studied and characterized. Again, recombinant SalG using crotonyl-CoA (commercially available) and chloro-crotonyl-CoA, will be prepared from methyl chlorocrotonate using a well-known CoA thioester synthesis. A spectrophotometric and HPLC assay will be used to monitor the oxidation of the nicotinamide cofactor (NADPH). Additionally, the single primary metabolic crotonyl-CoA reductase stro2345 will also be coned and over expressed, so its substrate specificity can be compared with SalG. This insight along with supported gene knockout experiments will reinforce SalG as a bona fide CCR involved in chloroethylmalonyl-CoA biosynthesis.

Still, in another aspect of the invention, the remaining gene products such as SalH and SalQ, as well as the missing dehydratase and carboxylase, will be studied and characterized substantially as described herein, including using known assays, which are abundant in the literature for their primary metabolic homologs.

Biosynthesis of the Novel Amino Acid β-hydroxycyclohexenylalanine

In addition to the 20 so-called proteinogenic amino acids whose incorporation into proteins is controlled by the information of the genetic code, non-proteinogenic amino acids can also be incorporated into peptide sequences by the described synthesis process. Non-proteinogenic amino acids are not genetically coded. Non-proteinogenic amino acids can also be classified as acidic, basic or neutral similar to proteinogenic amino acids.

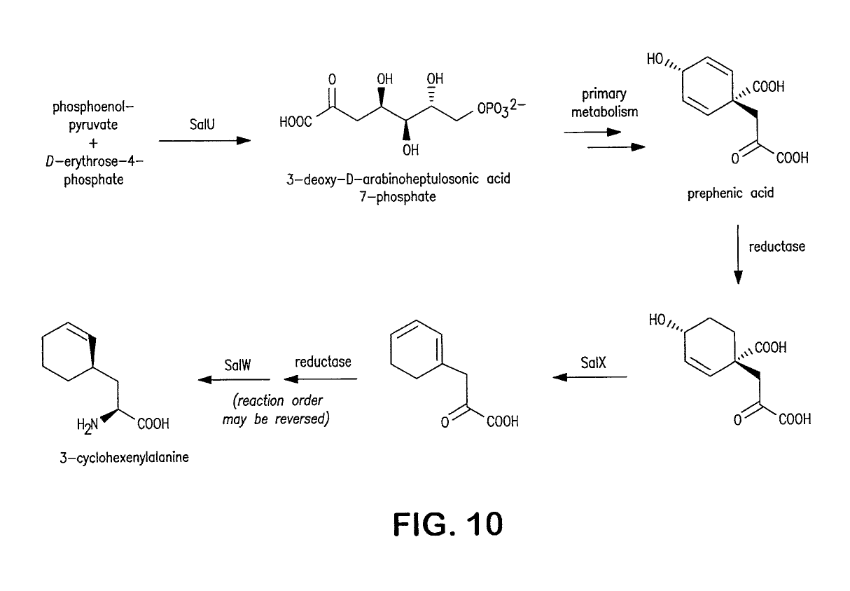

The second novel biosynthetic building block common to all salinosporamides is the non-proteinogenic amino acid β-hydroxycyclohexenylalanine (CHA). Preliminary data suggests that this amino acid is derived from a new shunt in the chorismate pathway as shown in FIG. 10. The key reaction is putatively catalyzed by the prephenate dehydratase-related enzyme SalX, which may catalyze the decarboxylative dehydration of dihydroprephenic acid to cyclohexadienylpyruvate (FIG. 10). Inactivation of the gene SalX by PCR-targeting is illustrated in FIG. 12, and the ensuing mutant is anticipated to no longer synthesize the known salinosporamides due to its inability to produce the amino acid. The organic extract by HPLC-MS will be analyzed as described herein and by cytotoxicity assays as described in more detail below. Thus, this mutant will provide an opportunity to 1) characterize the CHA biosynthetic pathway with synthetic intermediates; and 2) serve as a vehicle to introduce alternative amino acids into this region of the molecule in the engineering of new salinosporamide analogs.

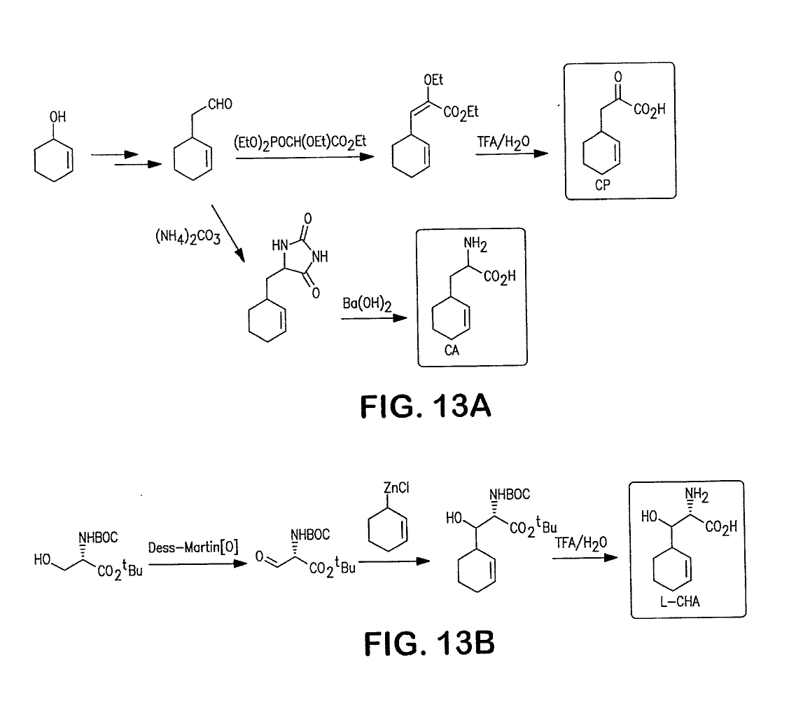

In another embodiment of the invention, CHA and its presumed biosynthetic intermediates as illustrated in FIG. 13, will be synthesized and characterized. First, 2-(cyclohex-2-enyl)acetaldehyde, which can be prepared from cyclohex-2-enol as previously described, will be converted in two steps to 2-(cyclohex-2-enyl)pyruvate (CP) based on the synthesis of related α-ketoacids from aldehydes (FIG. 13A). See, Barker et al. 2005 J. Med. Chem. 48, 4507-4510. The aldehyde can also serve as the starting point in the synthesis of the amino acid 3-(cyclohex-2-enyl)alanine (CA) via the hydantoin as previously described. See, Porter et al. 1968 J. Med. Chem. 11, 263-266. Lastly, L-CHA will be synthesized based on Corey's total synthesis of salinosporamide A. See. Reddy et al. 2004 J. Am. Chem. Soc. 126, 6230-6231. The Dess-Martin periodinane oxidation of protected L-serine and the attachment of the 2-cyclohexenyl unit via the allylic zinc reagent followed by deprotection should yield L-CHA (FIG. 13B). See, Miyake et al. 1992 Chem. Lett., 507-508. These syntheses are not designed to provide stereospecific products, but rather to quickly yield a mixture of isomers. For example, restoration of salinosporamide biosynthesis may occur upon feeding these unlabeled compounds to the salX mutant. In such case, efforts to separate the isomers or design new stereo-controlled syntheses will be required. The isomeric mixture will likely contain some of the unnatural isomers, which may serve as substrates and give rise to new salinosporamide derivatives. Furthermore, each of the syntheses allow for the introduction of stable isotopes for the preparation of labeled precursors for in vivo feeding experiments if desired.

A mutant deficient in CHA biosynthesis will be employed in a series of mutasyntheses to create structural diversity in the salinosporamides at the C-4 position, as this side chain is important for binding of the drug to the 20S proteasome, as well as likely conferring resistance in the S. tropica 20S proteasome subunit. Thus, ultimately the complete pathway to this new amino acid will be elucidated.

In Vivo Characterization of the Genes Encoding γ-lactam-β-lactone Assembly

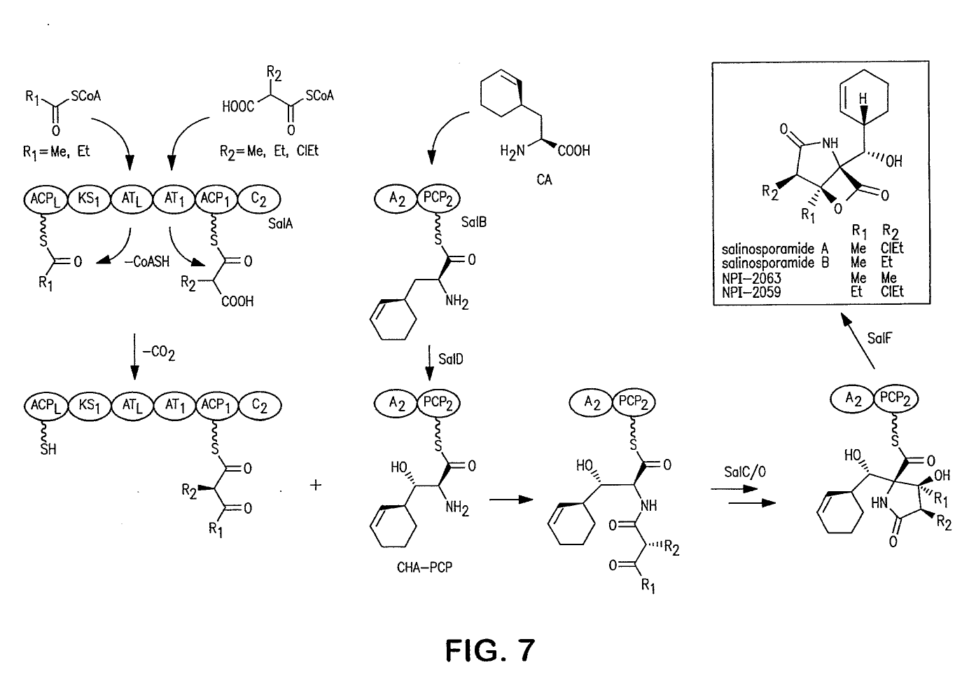

The putative SalB-bound linear product is converted to the γ-lactam-β-lactone by unprecedented PKS-NRPS biochemistry (FIG. 7). In order to elucidate and then manipulate this biochemistry, the protein-encoding genes need to be identified and characterized by mutagenesis. It is believed that SalB linear product undergoes an intramolecular condensation reaction to generate the γ-lactam, which then provides the SalB-bound alcohol substrate that gives rise to the offloaded β-lactone product. Candidate genes for the condensation reaction include the cyclase salO and the ketosynthase salC, while the α/β-hydrolase (thioesterase) salF is likely the β-lactonase. The respective mutants for salinosporamide biosynthesis will be characterized and genetically complementation studies to the salinosporamide-deficient mutants will be performed with the respective wild-type gene. For example, the β-lactone forming reaction using recombinant SalF as described herein are of interest.

In Vitro Analysis of the γ-lactam-γ-lactone Forming Enzymes

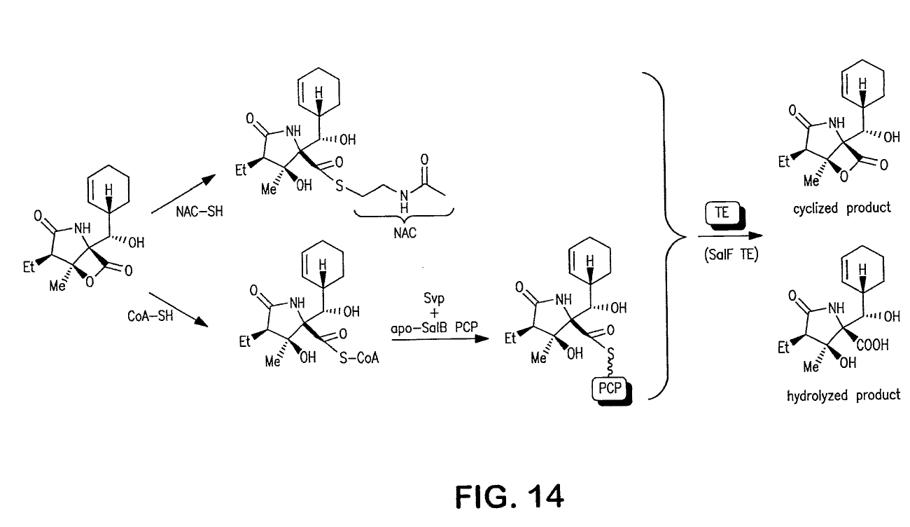

SalF is a stand-alone TE that likely catalyzes P-lactone formation and subsequent PCP off-loading. While most TEs are integrated domains in modular mega-synthetases, SalF is reminiscent of external TEIIs that serve editing functions by regenerating misprimed PCPs. Albeit rare, type II TEs also function to release fully elaborated products from their cognate carrier proteins, such as in coelichelin and phosphinothricin biosynthesis. SalF does not share significant sequence similarity to the other three stand-alone TEs present in the S. tropica genome such as stro2249, suggestive that it may have an alternative function as a β-lactonase. In order to evaluate the in vitro function of recombinant SalF, a series of γ-lactam substrates based on salinosporamide B will be performed. Also, due to the reactivity of the chloro group that could complicate the analysis, γ-lactam substrates based on salinosporamide B rather than salinosporamide A will be performed. The addition of NAC and coenzyme A to the electrophilic β-lactone should provide the respective esters (FIG. 14). The CoA thioester will be transferred to the isolated apo-PCP domain from SalB to yield salinosporamide B-S-PCP with the aid of the phosphopantetheinyl transferase Sfp120, Svp121, or one of the four S. tropica PPTases. The PPTase preparation of acyl-PCP substrates from apo proteins is now commonplace in the biosynthetic field and most recently employed with the microcystin synthetase. These three substrates will be incubated with SalF, and the anticipated products characterized by HPLC with authentic standards. The possibility that SalF is a bona fide β-lactonase is intriguing given that the β-lactone Orlistat (FIG. 3) is a suicide substrate of FAS TE. See, Kridel et al. 2004 Cancer Res. 64, 2070-2075.

If the in vivo and in vitro studies suggest that SalF catalyzes the hydrolysis of the γ-lactam but not the formation of the bicyclic γ-lactam-β-lactone, then there is likely another enzyme for this reaction. Like β-lactamase, the putative CoA ligase SalV, could similarly function to activate the carboxyl group in hydro salinosporamide B with ATP, which would then undergo an intramolecular displacement of AMP by the C-3 hydroxyl group to give the bicyclic γ-lactam-β-lactone. Recombinant SalF will then be tested to determine if it is an ATP-dependent β-lactonase. Still, other biosynthetic scenarios involving the cyclase SalO and the ketosynthase SalC are envisaged as biocatalysts for this novel biosynthetic reaction.

### Engineered Biosynthesis of Salinosporamides A and B

In another embodiment of the invention, there is provided a substantially purified salinosporamide composition, containing a first short chain fatty acid, a second short chain fatty acid, and a proteinogenic or non-proteinogenic amino acid, wherein the relative yield of the composition is substantially free of contaminants. The short chain fatty acids (SCFAs), which in the present context might be taken as having a carbon chain length of between 1 and 10, and preferably the carbon length is between 2 and 4, encompassing acetate, propionate and butyrate. Alternatively a broader range of fatty acids are contemplated by this invention, e.g., omega 3 fats (such as eicosapentaenoic acid). Proteinogenic and non-proteinogenic amino acids are also described herein.

The apparent relaxed substrate specificity of the elongating acyltransferase AT1 in the PKS SalA towards chloroethylmalonyl-CoA, ethylmalonyl-CoA and methylmalonyl-CoA is likely responsible for the corresponding structural diversity in the salinosporamide family at C-2 (FIG. 7). By controlling the biosynthesis of these different PKS elongating precursors, their relative cellular concentrations can be manipulated and modified (e.g., enhanced) through mutagenesis, thereby predictably redirecting the ratio of the salinosporamide analogs produced by fermentation. The present commercial production of salinosporamide A for clinical trials involves an expensive chromatographic step in which the minor (˜10%) analog salinosporamide B or other naturally occurring contaminants must be removed from the fermentation extract. Hence, S. tropica mutants with cleaner salinosporamide metabolic profiles impacts large-scale fermentation of the bacterium and subsequent isolation of the drug.

Given the novelty of the putative chloroethylmalonyl-CoA pathway, inactivation of various enzymes will and should inhibit this pathway. Hence, gene inactivation of the chlorinase salL will likely result in the loss of salinosporamide A synthesis and the concomitant overproduction of butyrate-derived salinosporamide B. However, genetic studies by knocking out salT (5′-ClDA phosphorylase) or salN (5-CIRP phosphatase), which also do not have homologs in the genome (Table 1), should also inhibit this pathway.

In order to retain salinosporamide A biosynthesis at the expense of salinosporamide B, butyryl-CoA biosynthesis is targeted. Comprehensive studies have shown that actinomycetes synthesize the PKS extender unit ethylmalonyl-CoA solely from the carboxylation of butyryl-CoA. Two major routes to butyryl-CoA have been delineated, and both are present in the S. tropica genome. The first is through valine-derived isobutyryl-CoA and is by catalyzed the coenzyme-B12-dependent isobutyryl-CoA mutase (ICM). The second pathway involves the reduction of acetate-derived crotonyl-COA by CCR. Sequence analysis of the S. tropica genome revealed two ICM-encoding genes (stro2349 and stro3005) and two CCR genes, one in the sal cluster (salG) and the second (stro2345) in a butyryl-CoA synthesis operon. Gene inactivation of SalG and characterization of recombinant SalG and Stro2345 will further determine SalG function. Also, PCR targeting studies (e.g., deleting part of the gene) the CCR stro2345 and the ICM genes will be performed, and characterization of the mutants for their effect on salinosporamide B production determined. The decrease in butyryl-CoA synthesis will likely result in an affiliated loss in salinosporamide B production, since the opposite effect (increased titers of salinosporamide B) was observed in wild-type feeding experiments with butyric acid.

The propionate-extender unit methylmalonyl-CoA for NPI-2063 biosynthesis as this salinosporamide product is produced at an acceptably low titer that does not impede the large scale purification of salinosporamide A. Further, feeding experiments with propionate increased the production of NPI-2063 in the wild-type organism. However, if the need arises to curtail this metabolite, gene inactivation and targeting of the methylmalonyl-CoA metabolic pathways as described herein.

In the present invention, the term “contaminant” or equivalent(s) thereof can be a inorganic or organic contaminant or material that should not be present in the salinosporamide A or analog thereof product as described herein. However, some organic material can be a valuable component, for example constituting the main product. The organic material may be of natural or synthetic origin, examples being proteins, sugars, amino acids, polyols and the like. As used herein, the term “substantially free” refers to less than about 20% contaminants in the resulting composition, which includes less than about 15%, less than about 10%, less than about 5% and less than about 2%.

In addition to utilizing the Sal gene cluster genes to make and produce recombinant salinosporamide A and analogs thereof substantially free of other naturally occurring organic or inorganic contaminants and/or materials, conventional methods for removing the contaminants/materials from aqueous systems include evaporative crystallization, cooling crystallization, reverse osmosis, extractive crystallization, ion exchange and the like. These and other means of removing contaminants is encompassed by the present invention.

### Engineered Biosynthesis of Unnatural Salinosporamide Analogs

The three side chains of the bicyclic γ-lactam-β-lactone core in the salinosporamide molecule are conveniently derived from separate biosynthetic precursors. Hence, targeting each section of the natural product independently through genetic engineering for the introduction of unnatural biosynthetic building blocks will be performed. A first study will eliminate the background production of the natural precursors and then feed synthetic analogs to the block mutants in a process called mutasynthesis. This strategy was employed previously in the generation of enterocin analogues.

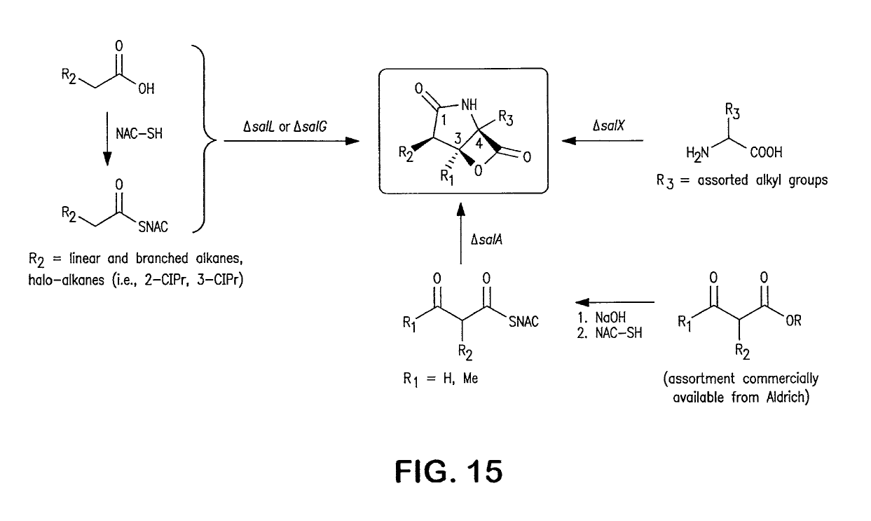

The crystal structure of the salinosporamide-bound yeast 20S proteasome shows that “C-2 substituents (R2 group) project into empty space” called the S2 pocket. See, Groll et al. 2006 J. Am. Chem. Soc. 128;5136-5141. This study further reported that it was “difficult to predict the optimal structure for the side chain based on the X-ray crystal structure of the complex”. The C-3 methyl (R1) group, on the other hand, interacts with a small pocket that can accommodate both a hydrogen (as in omuralide) and a methyl (as in the salinosporamides) group. Larger residues will “result in steric interactions that do not accommodate binding” and explains why NPI-2059, which has an ethyl group at C-3, does not inhibit the proteasome. See, Groll et al. 2006 J. Am. Chem. Soc. 128, 5136-5141; and Shah et al. 2002 J. Clin. Pharmacol. 54, 269-276. To effect changes selectively at C-2, assorted small chain carboxylic acids and acyl-SNACs will be fed to the ΔsalL and/or ΔsalG mutants (FIG. 15, Table 2) and monitor their incorporation as described above by HPLC-MS. The C-2 chloropropyl analog of salinosporamide A is of particular interest and is made by the addition of 5-chloropentanoate (or the SNAC thioester) to the ΔsalL mutant. This strategy assumes a broad substrate specificity of the SalA ATI, which appears to be the case based on the natural variability at C-2. However, changes into both the R1 and R2 groups simultaneously by the addition of diketide SNACs to a salA mutant in which the ketosynthase (KS) domain has been inactivated (FIG. 15, Table 2). Numerous β-ketothioesters are commercially available and can be converted to their corresponding NAC thioesters for administration. Ample precedence exists for the addition of PKS intermediates as NAC thioester analogs to PKS mutants in which a KS domain has been inactivated by site-directed mutagenesis. This approach may even allow for the introduction of a hydrogen atom at C-3 as in omuralide through the addition of malonic semi-aldehydes (FIG. 15).

TABLE 2
List of targeted sal genes for inactivation, their anticipated
phenotype, and engineering prospects through mutasynthesis

| | Predicted | Chemical | Targeted |
|---|---|---|---|
| Gene | phenotype | complement | mutasynthesis product(s) |
| salA | inactivation of KS domain will result | β-ketoacyl-SNACs | changes in R₁ and/or R₂ |
| | in loss of all sal production | | |
| salD | exclusive production of 5-deoxy | none planned | — |
| | salinosporamides | | |
| salG | reduction or loss of sal A | acyl-SNACs | changes in R₂ |
| | and/or sal B production | | |
| salL | exclusive elimination of sal A | 5′-XDA derivatives & | changes in R₂ |
| | | acyl-SNACs | |
| salX | elimination of known sals and | amino acids | changes in R₃ |
| | possible production of new | | |
| | derivatives in R₃ derived from | | |
| | native amino acid pool | | |

The amino acid-derived side chain R3 (FIG. 15, Table 2) is also a target of interest. On the basis of the X-ray crystal structure of the proteasome-salinosporamide complex, the C-5 substituents are involved in key interactions. See, Groll et al. 2006 J. Am. Chem. Soc. 128, 5136-5141. First, the cyclohexenyl ring interacts with the hydrophobic S1 binding pocket, which includes Val31, Met45 and Ala49. Second, the C-5 hydroxyl group is hydrogen bonded to the Thr21 amide nitrogen. These interactions are important factors in the docking of the drug to the S1 site of the proteasome to allow for the covalent addition to the N-terminal threonine residue to take place (FIG. 2). Side chain residues within the S1 binding pocket are sufficiently flexible as this pocket accommodates the larger cyclohexenyl side chain of the salinosporamides in place of the smaller isopropyl group in omuralide by shifting Met45 by 2.7 Å.

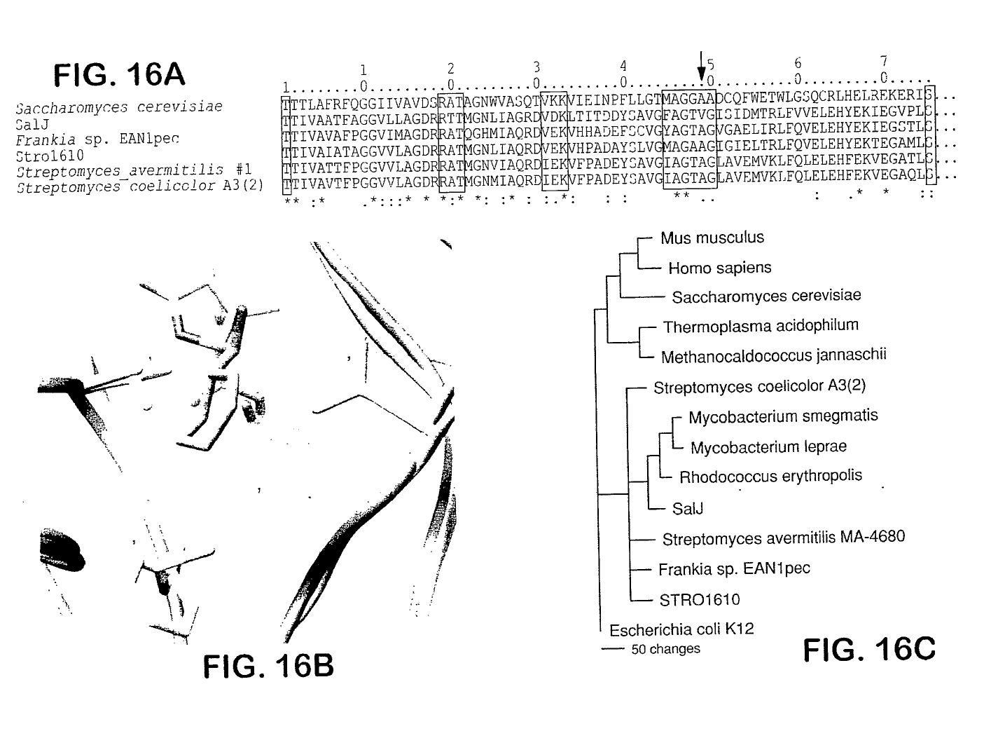

The rationale for desiring a change in the important C-5 substituents comes from the observation that the sal cluster contains an additional 20S proteasome β-subunit SalJ. The importance of this sal-specific proteasome subunit is discussed at length below. Conserved Ala49 residue is replaced with a larger valine residue, which would result in a smaller S1 binding pocket (FIG. 16). This point mutation in SalJ results in a salinosporamide-resistant proteasome in the bacterium that is expressed at the time of salinosporamide biosynthesis. If such a mutation evolved in S. tropica, then a similar mutation in humans could be devastating to salinosporamide therapy. This observation forms the basis behind the rationale for generating new analogs at C-5 with smaller substituents than cyclohexene. Thus, once a mutant deficient in CHA biosynthesis is generated (e.g., S. tropica ΔsalX described herein), the production of new salinosporamide analogs that incorporate natural or unnatural amino-acid residues can be tested. For example, adding to the culture medium of the mutant a variety of aliphatic amino acids including isoleucine, which is the equivalent amino acid present in omuralide. The β-hydroxyisoleucine derivative of salinosporamide A has in fact been chemically synthesized and shown to have comparative biological activity. Depending upon the outcome of these experiments, β-hydroxy amino acids may additionally be administered. A wide variety of amino acids are commercially available for these mutasynthesis experiments.

### Biological Assays

The new salinosporamide analogs resulting from methods described herein can be further assayed for biological activity in at least two assays. The first is the HCT116 cytotoxicity assay, which is a convenient in vitro, primary biological screen that can be used for rapid cytotoxicity testing during the chemical isolation process. The cytotoxic activity is determined by incubating the cells with test materials for about 72 hours in microtiter plates, and measuring cell viability. This cell line is routinely used in the screening of natural products and is available to us for this project. It has previously been reported that this in vitro assay is an excellent representative for in vivo activity and it is therefore the assay of choice during bioassay-guided fractionation.

Inhibition of the 20S proteasome will also be measured 20S for all new salinosporamides. The CHEMICON Proteasome Activity Assay Kit by Chemicon International provides a system that is designed to detect the chymotrypsin-like activity of the yeast 20S proteasome through the release of the fluorophore AMC (7-amino-4-methylcoumarin) from the specific substrate Suc-LLVY-AMC after cleavage. AMC can be detected using a fluorometer with a 380 nm excitation and a 460 nm emission filter. For inhibitor screening, the test sample is added to the assay plate and fluorescence read, looking for a reduction in proteasome activity. A negative control that does not contain test sample and a positive control with salinosporamide A (or lactacystin) will be run in each assay to ensure quality.

Although inhibition of the 20S proteasome is measured by the 20S proteasome cleavage of the cleavable product (fluorophore, AMC) from the cleavable substrate (Suc-LLVY-AMC), other cleavable product can be optionally detectably labeled with a moiety or moieties, including, for example, a fluorescent label, a luminescent label, chemiluminescent label, a colorigenic label, a radionuclide, or a paramagnetic label. In one aspect, the label comprises a fluorescence resonance energy transfer (FRET) pair, including a first fluorescent label and second fluorescent label with overlapping emission and excitation spectra, respectively; or a fluorescent label and a fluorescence quencher that quenches the fluorescence of the fluorescent label. For example, the cleavable substrate can be labeled with a first fluorescent label whereas the cleavable product can be labeled with a second fluorescent label with overlapping emission and excitation spectra. These and other methods of detectably labeling a compound or product or substrate are known in the art and are encompassed by the invention.

### Salinosporamide Resistance and 20S Proteasome Analysis

Bacterial proteasomes were thought to be nonexistent until the 20S proteasome was isolated and characterized from the nocardioform actinomycete Rhodococcus sp. strain N186/21.131 Subsequently, 20S proteasomes were found exclusively in other actinomycetes, including Mycobacterium, Streptomyces and Frankia. The 20S proteasome core is made from two α- and β-subunits referring to the homologous archaeal subunits first characterized from Thermoplasma. Electron micrographs from Rhodococcus and S. coelicolor show that the eubacterial structure is similar to achaebacterial and eukaryotic proteasomes, a stack of four rings forming a barrel shape with two juxtaposed rings of β-subunits flanked by two rings of α-subunits. The β-subunits of the 20S core are responsible for the proteolytic activity with the active sites confined to the interior compartment created via self-assembly. Mutagenesis studies have been conducted to create 20S proteasome-lacking Streptomyces, however, these have shown a non-essential role for cell viability. Upon genome analysis of S. tropica, a proteasome operon similar to the prcBA from other actinomycetes was discovered. In addition to what appears to be a fully intact proteasome operon stro1610-1611, S. tropica has a second β-subunit (salJ) present at one end of the sal gene cluster sandwiched between two transposases. Preliminary analysis of the amino acid sequence of SalJ revealed a potential role of resistance for this additional subunit. As described herein, the conserved Ala49 residue (numbered according to the yeast polypeptide) in the S1 binding pocket is present in Stro1610 (Ala49) yet replaced with a valine residue (Val49) in SalJ (FIG. 16A). Modeling studies suggest that the constriction of the S1 binding pocket in SalJ would prevent salinosporamide binding and result in a resistant 20S proteasome (FIG. 16B). This hypothesis will be tested by expressing recombinant β-subunits Stro1610 and SalJ and testing their relative proteolytic activities against the substrate, Suc-LLVY-AMC, in the presence of salinosporamides, lactacystin and other appropriate inhibitors as described herein. Stro1610 is inhibited while SalJ is not. Hence, whether inhibition can be reversed by the respective point mutants, V49A SalJ and A49V Stro1610, will help determine importance of the Ala→Val mutation. The validation of a resistant 20S proteasome in the bacterium S. tropica could impact the design of a second-generation salinosporamide.

The discovery, biosynthesis, mutasythesis, gene inactivation, knockout studies, in vitro and in vivo studies described herein are made possible because of the genome sequence of the marine actinomycete and to Salinispora arenicola. For instance, the genome sequences will allow comparison of the specificity of sal gene products with their primary metabolic counterparts (i.e., salG versus the single primary metabolic crotonyl-CoA reductase stro2345), locate posttranslational modifying enzymes important for the function of the pathway (i.e., there are four phosphopantetheinyl transferases (PPTases) in the genome), as well as probe for missing genes (i.e., one of the four acyl-CoA carboxylases present in the genome may also function as a chloroethylmalonyl-CoA carboxylase per FIG. 8).

### Kits

In another embodiment the invention, there is provided a pharmaceutical pack or kit containing one or more containers filled with any of the described Sal nucleic acids or encoding polypeptides, or nucleic acids or polypeptides derived from the Sal gene cluster described herein, or salinosporamide A and analogs thereof described herein, useful in treating, preventing or managing diseases or disorders associated with 20S proteasome activity and or function. In certain embodiments, the pharmaceutical pack or kit further contains a therapeutic or prophylactic agent useful in treating, preventing or managing diseases or disorders associated with 20S proteasome activity and or function.

In a preferred embodiment, the kit is useful in identifying agents which associate or interact with the described Sal nucleic acids or encoding polypeptides, or nucleic acids or polypeptides derived from the Sal gene cluster described herein, or salinosporamide A and analogs thereof described herein a purified enzyme or a biological sample.

The invention further provides a pack or kit containing one or more buffers, substrates or developers or detection systems, in one or more containers. Optionally associated with such container(s) can be a notice in the form prescribed by a governmental agency regulating the manufacture, use or sale of pharmaceuticals or biological products, which notice reflects approval by the agency of manufacture, use or sale for human administration. Further optionally associated with such container(s) can be instructions for use.

Although, the invention has been described with reference to the above examples, it will be understood that modifications and variations are encompassed within the spirit and scope of the invention. Accordingly, the invention is limited only by the following claims.

### Example 1

### Characterization of the Salinosporamide Biosynthetic Pathway

Inspection of the structures of the salinosporamides suggested that they originate from three biosynthetic building blocks, namely two variable small chain carboxylic acids and an amino acid. This hypothesis was largely based on the observation that this structural family had two carbon skeletal differences at C-2 (methyl, ethyl, chloroethyl) and C-3 (methyl, ethyl) (FIG. 1). Although the related lactacystin and its cyclization product omuralide were shown through feeding experiments to originate from valine-derived isobutyrate and leucine (Hollander, P. 2003 Prim. Care 30, 427-440; and Hadvary et al. 1991 J. Biol. Chem. 266, 2021-2027), it was hypothesized that this pathway was not compatible for the salinosporamides due to the variable nature of the lower half of the molecule.

To elucidate the identity of the salinosporamide building blocks, S. tropica, strain CNB476 was obtained. The strain, however, produced only about 1-5 mg salinosporamide A per liter and even less of the other analogs. This yield was not acceptable for labeling experiments, so XAD-7 resin was added to the culture medium about one day after inoculation, harvested after about 5 days and eluted the resin with acetone and further purified by column chromatography, to yield an extract that was about ˜80% pure salinosporamides or about 60-80 mg salinosporamide A per liter. Nereus reports titers of 450 mg/L, however, their method was performed through strain selection and improvements in fermentation. These data suggest that salinosporamide production is regulated by a feedback mechanism. This approach provided a huge increase in titer, as well as a very simple isolation protocol.

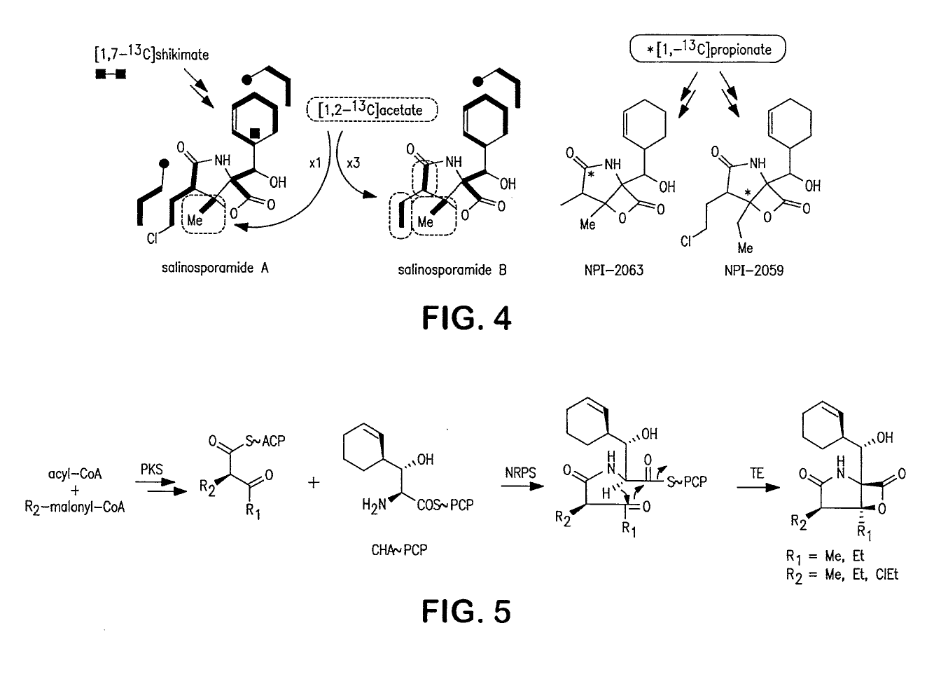

A large series of feeding experiments with stable isotopes to probe the biosynthesis of salinosporamides A and B, NPI-0059 and NPI-0062, respectively were performed. Initial feeding experiments with [U-13C6]glucose, [1,2-13C2]acetate, [1-13C]butyrate, [1-13C]phenylalanine, and [1,7-13C2]shikimate were particularly illuminating as they confirmed that the salinosporamides are biosynthesized from three metabolic building blocks. The experiments also suggested an unprecedented PKS-NRPS hybrid pathway and outlined the formation of a novel amino acid unit via a new shunt in the phenylalanine pathway. Surprisingly, the experiments also showed that the chloroethyl and ethyl side chains in salinosporamides A and B, respectively, originate from two completely different precursors (FIG. 4). While the ethyl side chain in salinosporamide B is indeed derived from butyrate, the chloroethyl side chain in salinosporamide is derived from a sugar, as NMR analysis of [U-13C6]glucose-enriched salinosporamide A confirmed two labeling patterns indicative of a tetrose. The identity of this sugar-derived unit was unknown until the biosynthetic gene cluster, discussed in more detail below, was characterized. Further feeding experiments with [1-13C]propionate confirmed the modular nature of the C-2 side chain in NPI-2063 as well as demonstrating that propionate can replace the acetate-derived starter unit in NPI-2059.

Together these incorporation experiments suggested a biosynthetic pathway in which acetyl-CoA and a substituted malonyl-CoA molecule are condensed by a PKS to generate a β-keto thioester, which then reacts with the non-proteinogenic amino acid L-β-hydroxycyclohexenylalanine (CHA) to give the hybrid PKS-NRPS product (FIG. 5).

### Example 2

### Sequence of the Salinispora tropica Genome and Analysis of the Sal Gene Cluster

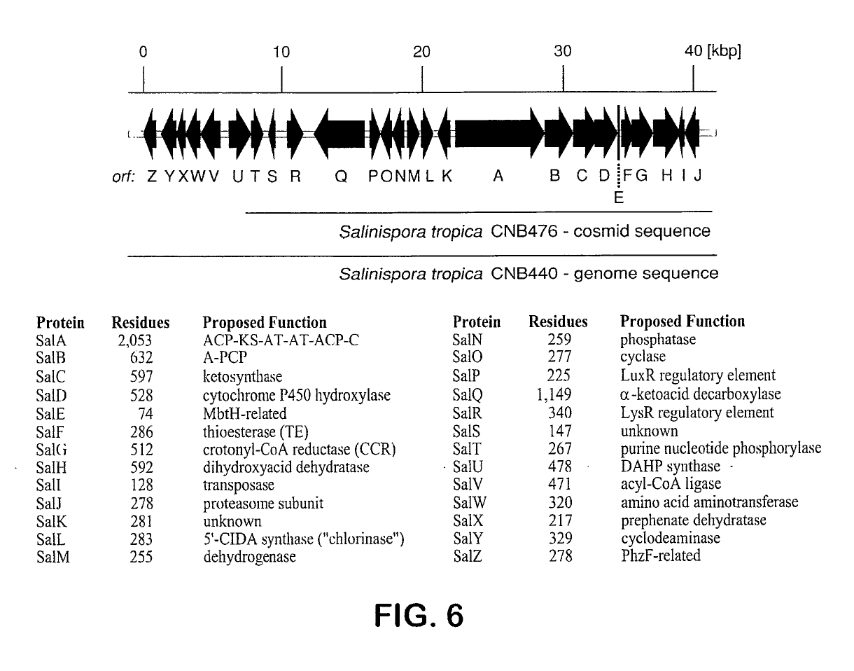

The putative salinosporamide biosynthetic gene cluster (sal) in two S. tropica isolates was sequenced using two different methods. In S. tropica CNB476, a traditional approach using PCR amplified biosynthetic genes, characteristic of the pathway (FIG. 5), were based on the labeling studies. PKS, NRPS, P450 (amino acid β-hydroxylase), and crotonyl-CoA reductase (CCR) gene fragments and correlated these probes to a single 32-kb pOJ446 cosmid clone that was subsequently sequenced. This sequence provided the majority of the gene cluster, yet it was not complete, as the proposed sal cluster was larger than the cloned insert (FIG. 6). The second approach involved S. tropica CNB440 to sequence its entire genome. This information is described on the world wide web at jgi.doe.gov/sequencing/why/CSP2006/salinispora.html, which is incorporated herein by reference in its entirety. The completed genome was released to the public in August 2006. At present 4,547 candidate protein-coding genes have been annotated, of which about 26% are annotated as hypothetical. Inspection of the draft sequence revealed 17 natural product biosynthetic gene clusters, including the previously sequenced sal cluster from CNB476. Bioinformatic analysis of all clusters revealed that the sal cluster was the only logical biosynthetic gene cluster that could be involved in salinosporamide biosynthesis based on the isotope experiments described above. A mixture of polypeptides have been identified including, type I, II and III PKSs, NRPSs, hybrid PKS-NRPSs, siderophore pathways, terpenoid pathways, and even an aminocyclitol. More importantly, this data set provides the exact location of important secondary metabolic pathway-associated genes whose products provide common biosynthetic precursors and enzyme cofactors as well as perform key post-translational protein modifications.

Analysis of the sal cluster allowed refines the biosynthetic pathway which was initially based on the isotope experiments. The sequence was largely consistent with the scheme outlined in FIG. 5 and strongly suggested an unexpected and unprecedented chlorination pathway. The bimodular PKS SalA contains six domains, which are similarly organized as in the soraphen PKS SorA.60 SalA is involved in the selection, attachment and condensation of acetyl-CoA and a substituted malonyl-CoA molecule to generate a β-ketothioester intermediate (FIG. 7). Sequence analysis of the two acyltransferases suggested that ATL is selective for acetyl-CoA whereas the selectivity of AT1 is much less clear. Based on the biosynthetic model in FIG. 7, AT1 likely prefers the new PKS extender unit chloroethylmalonyl-CoA, which provides the postulated tetrose-derived unit of salinosporamide A; yet has broad specificity toward other substituted malonyl extender units. The product of this PKS does not undergo β-reduction given the absence of reductive machinery in the PKS. The NRPS extension module resides on two proteins, SalA (C-terminal C domain) and SalB (A-PCP didomain). The aliphatic amino acid L-3-(cyclohex-1′R-2′-enyl)-alanine (CA) is activated and transferred to the holo-PCP domain of SalB, hydroxylated by SalD, and then condensed with the SalA diketide by action of the SalA C domain. The SalB-bound linear product must then undergo a formal loss of the amino acid α-hydrogen as part of a condensation with the ketone carbonyl to generate the γ-lactam ring intermediate, and it is proposed that this unprecedented PKS-NRPS chemistry may be catalyzed by the type II PKS homologous cyclase SalO and/or the KS SalC. The thioesterase SalF is then postulated to off-load the SalB-bound product directly as the β-lactone product. The postulated formation of the bicyclic ring constitutes truly novel biochemistry associated with a PKS-NRPS system and thus will be mechanistically probed in this application.

### Example 3

### Characterization of a Novel Chlorination Pathway

Identification of the sal biosynthetic gene cluster was instrumental in illuminating an unexpected and unprecedented chlorination pathway in nature to the chlorinated tetrose-derived unit in salinosporamide A. Surprisingly, none of the typical oxygen-based chlorinating enzymes, such as heme- and vanadium-dependent haloperoxidases that use H₂O₂ as a co-substrate or flavin- and non-heme iron-dependent halogenases that utilize dioxygen, were encoded by genes in the sal cluster. This general oxidative strategy has only recently been clarified and is routinely employed in nature with chloride, bromide and iodide ions to generate a reactive electrophile (X+) or radical (X.) to deliver to electron-rich and electron-poor systems, respectively. The direct nucleophilic attack of the halide X-upon carbon electrophiles, on the other hand, is extremely rare in nature. Just two examples have been reported, and both utilize the cofactor S-adenosyl-L-methionine (SAM). Methyl halide transferases are uncommon enzymes that use a halide ion to displace S-adenosylhomocysteine from the cofactor to generate methyl halides. A better understood system with described biological relevance involves the recently discovered enzyme fluorinase from Streptomyces cattleya, which catalyzes the nucleophilic displacement of methionine from SAM with F— ion to generate 5′-fluoro-5′-deoxyadenosine (5′-FDA) as the first step in the biosynthesis of fluoroacetic acid and fluorothreonine. See O'Hagan et al. 2002 Nature 416, 279-279; and Deng et al. 2004 Nat. Prod. Rep. 21, 773-784. The fluorinase homologous gene salL (35% identity) is centrally located in the sal gene cluster (FIG. 6).

Examination of the complete sal cluster revealed the makings of a new metabolic pathway for the conversion of SAM via 5′-chloro-5′-deoxyadenosine (5′-ClDA) to the postulated PKS extender unit chloroethylmalonyl-CoA (FIG. 8). This novel chlorinated extender unit provides the biosynthetic equivalent of a 4-chlorobutyryl unit to the PKS SalA in the enzymatic synthesis of salinosporamide A (FIG. 7). The 13C-labeled glucose incorporation pattern in the tetrose-derived unit in salinosporamide A is consistent with the ribose unit of SAM being the ultimate precursor. It appears, therefore, that S. tropica has evolved an exquisite pathway that borrows a number of genes from primary metabolism for the assembly of chloroethylmalonyl-CoA (FIG. 8).

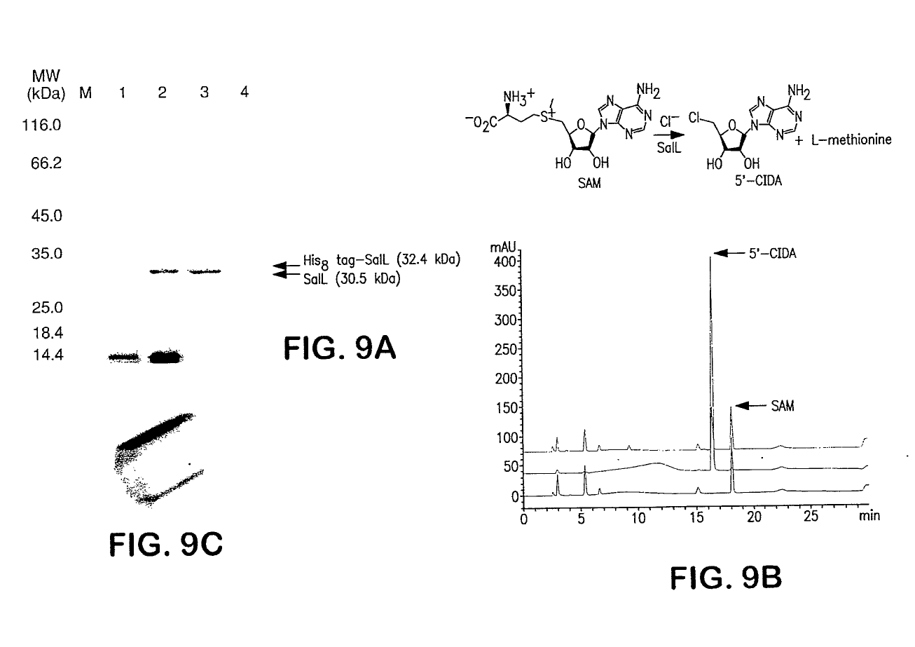

The fluorinase homolog SalL commits SAM from the primary metabolic pool to this secondary metabolic pathway. To test this hypothesis, salL was first cloned into the pHIS8 expression vector and over expressed the octahistidyl-tagged recombinant protein in E. coli BL21 (DE3). The recombinant N-terminal His8-tagged protein (˜60 mg/L culture) was purified by Ni2+-affinity chromatography and had a mass of ˜32 kDa corresponding to SDS-PAGE, which was in close agreement with the value 32,422.8 Da calculated for the tagged protein (FIG. 9A). Incubation with SAM and NaCl established that SalL was indeed a bona fide 5′-chloro-5′-deoxyadenosine (5′-ClDA) synthase as confirmed by HPLC analysis with authentic standards (FIG. 9B). Recently, it was shown that fluorinase from S. cattleya also operates in vitro as a chlorinase, albeit with 120:1 rate preference for F— over Cl—. This chlorinating activity was only observed in coupled enzyme assays designed to shift the equilibrium of the reaction toward 5′-ClDA. SalL, on the other hand, did not require such manipulation, as the organochloride product was indeed preferred.

Given the novelty of this enzyme, the three-dimensional structure by X-ray crystallography was determined. The His8 tag was removed by thrombin cleavage, and the protein further purified by gel filtration (FIG. 9A, lane 4). The protein is very well behaved, and crystals were readily obtained from different conditions using the hanging drop vapor diffusion method (an example is shown in FIG. 9C). The elucidation of its three-dimensional structure is on going. Data sets on SalL soaked with the product 5′-ClDA at the Advanced Light Source in Berkeley, Calif. at ˜2.0 Å resolution and anticipate solving its structure.

It is likely that the SalL product 5′-ClDA is then converted in a series of reactions catalyzed by pathway-specific enzymes evolved from primary metabolic homologs to 4-chlorobutyryl-CoA in which the purine is eliminated from 5′-ClDA to give 5-chloro-5-deoxyribose-1-phosphate (5-CIRP) and then C-1 of the pentose is eventually lost to yield the tetrose-derived unit (FIG. 8). Again, it is this reactive chloroethyl group that is essential to the irreversible binding of the drug to the proteasome (FIG. 2).

### Example 4

### Biosynthesis of the Novel Non-Proteinogenic Amino Acid L-3-Cyclohexenylalanine

The feeding experiment with [U-13C6]glucose suggested a pathway to the amino acid moiety that paralleled phenylalanine biosynthesis in which the cyclohexyl ring never becomes symmetrical due to the observed nonsymmetrical labeling pattern in the product salinosporamide A (see FIG. 4). It was confirmed that while the amino acid was indeed derived from chorismate through feeding experiments with [1,7-13C2]shikimate and [U-13C10]chorismate, it did not originate from phenylalanine itself as [1-13C]phenylalanine was not incorporated. Hence the pathway illustrated in FIG. 10 in which either chorismate or prephenate is shunted into the cyclohexenylalanine pathway by the formal reduction of the pro-R C2′-C3′ double bond of prephenate was proposed. A prephenate dehydratase homolog would then catalyze the decarboxylative dehydration to the diene, which would then be processed to the amino acid by further double bond reduction and transamination. The sal cluster harbors many of the desired genes for such a pathway, including a copy of the initiating shikimic acid pathway enzyme DAHP synthase (SalU), a prephenate dehydratase homolog (SalX), and an aliphatic L-amino acid aminotransferase (SalW). This pathway represents a new branch in the chorismate pathway.

### Example 5

### Gene Inactivation of Salinospora Gene Cluster

In order to clarify and harness the potential of the sal gene cluster (as well as the entire S. tropica genome), a genetics system in S. tropica for making genetic knockouts was established. As a proof of principle, a pKC1132-based plasmid (pAEM3) in which was cloned an internal 2.6-kbp PstI fragment from the 6.1-kbp salA PKS gene was produced. This suicide vector was introduced by conjugation from E. coli ET12567/pUZ8002 into S. tropica CNB440, yielding about 100 exconjugants via single-crossover homologous recombination. Southern blot analyses of two independent exconjugants confirmed the insertion of pAEM3. Mutant and parental strains were cultured in the A1 production medium, and HPLC/MS analyses show abolition of salinosporamide production in the mutant (FIG. 11). This is further confirmation, beyond genome sequence data, that the sal cluster indeed encodes salinosporamide biosynthetic enzymes.

Gene inactivation via a single crossover as described above represents a relatively quick and classical way to disrupt genes in actinomycetes and was used here to probe methods of introducing DNA into S. tropica for the first time. Although mutants created in this way are very useful for gross phenotypic characterization, they can revert to wild-type by excision of the vector and have thus the potential to be unstable. Moreover, polar effects on downstream genes have been observed. Therefore, adapting a PCR-based mutagenesis approach for Salinispora that was first used with Streptomyces coelicolor and now gaining popularity in the biosynthetic community as an efficient gene replacement strategy is ongoing. Importantly, with a genetics system now in hand, there are a multitude of opportunities to explore various aspects of salinosporamide biosynthesis and biology.

For example, a PCR-targeting strategy for rapid gene knockouts as proposed in FIG. 12 has been established. SalL (chloridase), SalG (putative chlorocrotonyl-CoA reductase) and SalD (cytochrome P450) have been inactivated. See Table 2. Inactivation of SalL and salG both led to mutants that completely lost their ability to synthesize salinosporamide A while still maintaining a normal production of the non-halogenated salinosporamides. In contrast, the SalD knockout led to new 5-deoxysalinosporamides (FIG. 18). Chemical complementation of the ΔsalL mutant with 5′-chloro-5′-deoxyadenosine (5′-ClDA) restored salinosporamide A production and thereby confirmed the biological function of the chloridase SalL as originally proposed in FIG. 8.

### Example 6

### Crystal Structure of a Salinospora Analog

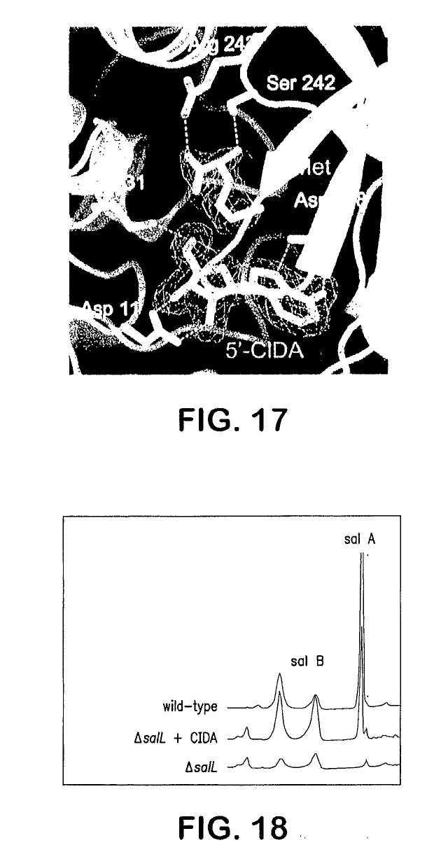

The X-ray crystal structure of the homotrimeric chloridase SalL was accomplished at 1.55 Å resolution with products L-methionine and 5′-ClDA (FIG. 17). A number of point mutants (G131S, Y70Y, W129F, double mutant G131S/Y70T) were also generated to probe halide specificity and mechanism. The structure of the Y70T mutant was solved at 1.8 Å resolution bound with substrates S-adenosylmethionine and chloride. With these structures and several other structures to be determined, there is an efficient system to systematically characterize the mechanism of this and other enzymes.

### Example 7

### Salinosporamide J Confers 20S Proteasome Resistance

To demonstrate that salinosporides synthesized and discovered as above can affect 20S proteasome degradative activity in vitro, experiments were conducted using the 20S Proteasome Activity Assay (Chemicon) in the presence or absence of a proteasome inhibitor.

The ubiquitin-proteasome pathway involves the 26S proteasome, which is formed by the barrel-shaped 20S proteasome (about 700 kDa) and two 19S (about 700 kDa) regulatory complexes on each end of the 20S proteasome. The 20S Proteasome Activity Assay measures 20S proteasome activity by detecting cleavage of substrates. For example, the 20S Proteasome Activity Assay provides detection of the cleavage of LLVY-flourophore, 7-Amino-4-methylcoumarin (AMC) substrate (Meng et al. 1999 PNAS USA 96:10403-08). Cleavage of the free flourophore, 7-Amino-4-methylcoumarin (AMC) from LLVY can be detected and quantified using a 380/460 nm filter set in a flourmeter. The assay is performed in the presence or absence of a proteasome inhibitor, e.g., SalA and/or Lactacystin, for screening purposes. The protocol for using the 20S Proteasome Activity Assay is substantially as described in the product sheet provided in the kit from Chemicon and is incorporated herein in its entirety by reference.

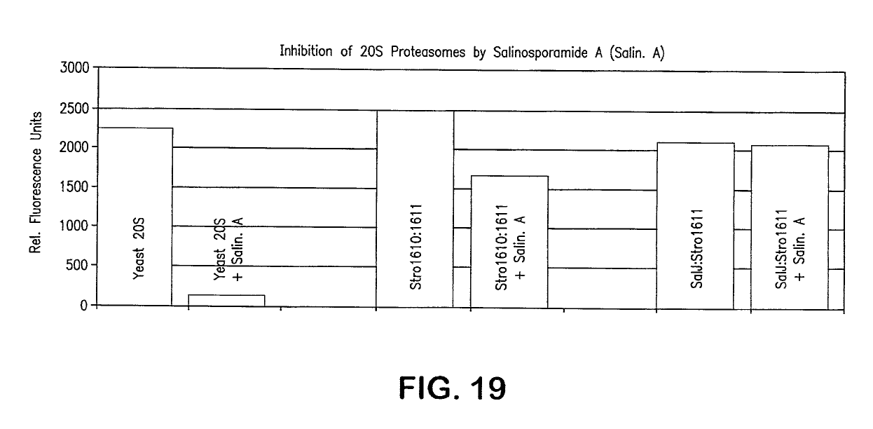

The present invention describes the biosynthesis of SalJ (FIG. 16). 20S proteasomes (about >700 kDa) derived from recombinant α-[Stro1611 (27.5 kDa)] and β1-[Stro1610 (28.8 kDa)] or β2-[SalJ (29.4 kDa)] subunits from S. tropica were assembled with a stoichiometry of α7β7β7α7. The SalJ β-subunit contains the Ala→Val mutation at residue 49. The assembled 20S proteasome demonstrated that both chromosomal SalJ β-subunits were proteolytically active when fully assembled with the α-subunits. Preliminary experiments demonstrated that while the yeast and S. tropica 20S proteasomes were inhibited with salinosporamide A (FIG. 19), the assembled 20S proteasome from recombinant subunits containing the SalJ β-subunit was not inhibited by salinosporamides A (FIG. 19). FIG. 17 shows that yeast 20S proteasomes, in the presence of SalA had significantly reduced relative levels of fluorescence of the free AMC (FIG. 19, far left, short bar) as compared to the yeast 20S proteasome sample without SalA (FIG. 19, far left, taller bar). Similarly, the S. tropica 20S proteasome (FIG. 19, middle two bars) were also inhibited in the presence of SalA. Yet, the assembled-recombinant 20S proteasome with the SalJ β-subunit (far right two bars) confers resistance to at least SalA as there was no substantial measurable difference in the level of fluorescence of the free AMC (FIG. 19) in the presence or absence of SalA. Hence, the SalJ β-subunit incorporated into the assembled-recombinant 20S proteasome confers resistance for the proteasome. Thus, other salinosporamide analogs and/or derivatives discovered from the biosynthesis studies described herein may also be capable of conferring 20S proteasome inhibition.

Although the present process has been described with reference to specific details of certain embodiments thereof in the above examples, it will be understood that modifications and variations are encompassed within the spirit and scope of the invention. Accordingly, the invention is limited only by the following claims.

1. A substantially purified salinosporamide composition, comprising:
(a) a first short chain fatty acid;
(b) a second short chain fatty acid; and
(c) a proteinogenic or non-proteinogenic amino acid,
wherein the relative yield of the composition is substantially free of contaminants.

2. The substantially purified salinosporamide composition of claim 1, wherein:
(i) the first short chain fatty acid is an acetate;
(ii) the second short chain fatty acid is a 5′-chloro-5′-deoxyadenosine (ClDA)-derived intermediate; and
(iii) the amino acid is a non-proteinogenic amino acid.

3. The composition of claim 1, wherein the first or second short chain fatty acid comprises a carbon length between 1 and 10.

4. The composition of claim 1, wherein the first short chain fatty acid is acetate.

5. The composition of claim 1, wherein the second short chain fatty acid is a 5′-chloro-5′-deoxyadenosine (ClDA)-derived intermediate.

6. The composition of claim 1, wherein the amino acid is a non-proteinogenic amino acid.

7. The composition of claim 1, wherein the non-proteinogenic amino acid is cyclohexenylalanine.

8-11. (canceled)

12. The composition of claim 5, wherein the ClDA-derived intermediate is derived from S-adenosyl methionine.

13. The composition of claim 1, wherein the non-proteinogenic amino acid is any amino acid derived from the shikimic acid pathway as set forth in FIG. 10.

14. (canceled)

15. The composition of claim 1, wherein the composition is substantially free of salinosporamide B.

16. A method of identifying a 20S proteasome resistant salinosporamide or an analog thereof, comprising:
a contacting a 20S proteasome and a salinosporamide or an analog thereof in the presence of a cleavable substrate, wherein the substrate comprises a cleavable product, wherein the 20S proteasome has proteasome activity, wherein the proteasome cleaves the cleavable product from the substrate in the presence or absence of a 20S proteasome inhibitor; and
(b) detecting change in the proteasome activity by measuring the cleavable product, thereby identifying a proteasome resistant salinosporamide and/or analog thereof.

17. The method of claim 16, wherein the salinosporamide or analog thereof comprises:
(a) a first short chain fatty acid:
(b) a second short chain fat acid; and
(c) a proteinogenic or non-proteinogenic amino acid,
wherein the relative yield of the salinosporamide is substantially free of contaminants.

18. The method of claim 16, wherein the salinosporamide or analog thereof is salinosporamide J.

19. The method of claim 16, wherein the cleavable product is detectably labeled.

20-21. (canceled)

22. The method of claim 16, wherein the cleavable product comprises a fluorogenic peptide substrate.

23. The method of claim 22, wherein the flourogenic compound is 7-Amino-4-methylcoumarin (AMC).

24. The method of claim 16, wherein the 20S proteasome inhibitor is selected from the group consisting of peptide aldehydes, peptide vinyl sulfones, and peptide epoxyketones.

25. The method of claim 16, wherein the 20S proteasome inhibitor is selected from the group consisting of salinosporamide A, lactacystin, Ac-PRLN-vs, ajoene, Acetyl-Leu-Leu-NorLeucinal (Ac-LLN-al), Gold(III) dithiocarbonate, bortezomib, NP1-0052, PS-341, PS-519 and MG-132.

26. The method of claim 16, wherein the 20S proteasome inhibitor is salinosporamide A or a derivative thereof.

27. The method of claim 16, wherein the 20S proteasome inhibitor is lactacystin or a derivative thereof.

28. A method of producing a salinosporamide from a transformed bacterium, comprising:
a introducing a transgene which disrupts or interferes with expression of salinosporamide; and
cross-breeding transgene-positive progeny with each other to obtain further transgene-positive progeny.

29. The method of claim 28, wherein the transgene comprises a salinosporamide gene cluster nucleic acid.

30. The method of claim 28, wherein the transgene comprises a salinosporamide A or nucleic acid or an analog thereof.

31. The method of claim 28, wherein the transgene is polyketide synthase (PKS) nucleic acid.

32. The method of claim 28, wherein the transgene is an anti-sense nucleic acid.

33. A method of manufacturing a substantially pure salinosporamide derivative comprising, combining a first short chain fatty acid, a second short chain fatty acid, and a proteinogenic or non-proteinogenic amino acid, wherein the relative yield of the composition is substantially free of contaminants.

34. The method of of claim 33, wherein the first short chain fatty acid is an acetate, the second short chain fatty acid is a 5′chloro-5′deoxyadenosine (ClDA)-derived intermediate, and the amino acid is a non-proteinogenic amino acid.

35. The method of claim 33, wherein the first or second short chain fatty acid comprises a carbon length between 1 and 10.

36. The method of claim 33, wherein the first short chain fatty acid is acetate.

37. The method of claim 33, wherein the second short chain fatty acid is a 5′-chloro-5′-deoxyadenosine (ClDA)-derived intermediate.

38. The method of claim 33, wherein the amino acid is a non-proteinogenic amino acid.

39. The method of claim 33, wherein the non-proteinogenic amino acid is cyclohexenylalanine.

40-43. (canceled)

44. The method of any of claim 37, wherein the ClDA-derived intermediate is derived from S-adenosyl methionine.

45. The method of claim 38, wherein the non-proteinogenic amino acid is any amino acid derived from the shikimic acid pathway as set forth in FIG. 10.

46. (canceled)

47. The method of claim 33, wherein the composition is substantially free of salinosporamide B.

48-52. (canceled)

53. A method of producing high-titer recombinant salinosporamide A or an analog thereof, comprising:
(a) simultaneously co-infecting a cell with a nucleic acid encoding salinosporamide A or an analog thereof operably linked to a promoter;
(b) incubating and growing the cell under suitable conditions; and
(c) collecting the salinosporamide A or an analog thereof from the cell of step (b), thereby producing a high-titer.
